(19) United States
(12) Patent Application Publication
(10) Pub. No.: US 2011/0070582 A1
(43) Pub. Date: Mar. 24, 2011

(54) Gene Expression Profiling for Predicting the Response to Immunotherapy and/or the Survivability of Melanoma Subjects

(75) Inventors: Danute M. Bankaitis-Davis, Longmont, CO (US); Lisa Siconolfi, Westminster, CO (US); Kathleen Storm, Longmont, CO (US); Karl Wassmann, Dover, MA (US)

(73) Assignee: Source Precision Medicine, Inc. d/b/d Source MDX, Boulder, CO (US)

(21) Appl. No.: 12/792,443

(22) Filed: Jun. 2, 2010

Related U.S. Application Data
(63) Continuation-in-part of application No. PCT/US2009/063138, filed on Nov. 3, 2009.
(60) Provisional application No. 61/110,786, filed on Nov. 3, 2008.

(51) Int. Cl. C12Q 1/68

(52) U.S. Cl. 435/6

(57) ABSTRACT

A method is provided in various embodiments for determining a profile data set for predicting the response top immunotherapy and or survivability of a subject with melanoma based on a sample from the subject, wherein the sample provides a source of RNAs. The method includes using amplification under measurement conditions that are substantially repeatable for measuring the amount of RNA corresponding to at least 2, 3 or 4 constituents according to the gene models shown in Tables 2-3, 4-6 and 9.

### REFERENCE TO RELATED APPLICATIONS

This application is a continuation in part and claims priority to, PCT Application Serial No. PCT/US2009/063138, filed Nov. 3, 2009 which claims the benefit of U.S. Provisional Application No. 61/110,786, filed Nov. 3, 2008, the contents of each are hereby incorporated by reference in its entirety.

### FIELD OF THE INVENTION

The present invention relates generally to the identification of biological markers of melanoma-diagnosed subjects capable of predicting primary end-points of melanoma progression. More specifically, the present invention relates to the use of gene expression data in the prediction of the response to immunotherapy, survivability and/or survival time of melanoma-diagnosed subjects.

### BACKGROUND OF THE INVENTION

Skin cancer is the growth of abnormal cells capable of invading and destroying other associated skin cells. Skin cancer is the most common of all cancers, probably accounting for more than 50% of all cancers. Melanoma accounts for about 4% of skin cancer cases but causes a large majority of skin cancer deaths. The skin has three layers, the epidermis, dermis, and subcutis. The top layer is the epidermis. The two main types of skin cancer, non-melanoma carcinoma, and melanoma carcinoma, originate in the epidermis. Non-melanoma carcinomas are so named because they develop from skin cells other than melanocytes, usually basal cell carcinoma or a squamous cell carcinoma. Other types of non-melanoma skin cancers include Merkel cell carcinoma, dermatofibrosarcoma protuberans, Paget's disease, and cutaneous T-cell lymphoma. Melanomas develop from melanocytes, the skin cells responsible for making skin pigment called melanin. Melanoma carcinomas include superficial spreading melanoma, nodular melanoma, acral lentiginous melanoma, and lentigo maligna.

Basal cell carcinoma affects the skin's basal layer, the lowest layer of the epidermis. It is the most common type of skin cancer, accounting for more than 90 percent of all skin cancers in the United States. Basal cell carcinoma usually appears as a shiny translucent or pearly nodule, a sore that continuously heals and re-opens, or a waxy scar on the head, neck, arms, hands, and face. Occasionally, these nodules appear on the trunk of the body, usually as flat growths. Although this type of cancer rarely metastasizes, it can extend below the skin to the bone and cause considerable local damage. Squamous cell carcinoma is the second most common type of skin cancer. It is a malignant growth of the upper most layer of the epidermis and may appear as a crusted or scaly area of the skin with a red inflamed base that resembles a growing tumor, non-healing ulcer, or crusted-over patch of skin. It is typically found on the rim of the ear, face, lips, and mouth but can spread to other parts of the body. Squamous cell carcinoma is generally more aggressive than basal cell carcinoma, and requires early treatment to prevent metastasis. Although the cure rate for both basal cell and squamous cell carcinoma is high when properly treated, both types of skin cancer increase the risk for developing melanomas.

Melanoma is a more serious type of cancer than the more common basal cell or squamous cell carcinoma. Because most malignant melanoma cells still produce melanin, melanoma tumors are often shaded brown or black, but can also have no pigment. Melanomas often appear on the body as a new mole. Other symptoms of melanoma include a change in the size, shape, or color of an existing mole, the spread of pigmentation beyond the border of a mole or mark, oozing or bleeding from a mole, and a mole that feels itchy, hard, lumpy, swollen, or tender to the touch.

Melanoma is treatable when detected in its early stages. However, it metastasizes quickly through the lymph system or blood to internal organs. Once melanoma metastasizes, it becomes extremely difficult to treat and is often fatal. Although the incidence of melanoma is lower than basal or squamous cell carcinoma, it has the highest death rate and is responsible for approximately 75% of all deaths from skin cancer in general.

Cumulative sun exposure, i.e., the amount of time spent unprotected in the sun is recognized as the leading cause of all types of skin cancer. Additional risk factors include blond or red hair, blue eyes, fair complexion, many freckles, severe sunburns as a child, family history of melanoma, dysplastic nevi (i.e., multiple atypical moles), multiple ordinary moles (>50), immune suppression, age, gender (increased frequency in men), xeroderma pigmentosum (a rare inherited condition resulting in a defect from an enzyme that repairs damage to DNA), and past history of skin cancer.

Treatment of skin cancer varies according to type, location, extent, and aggressiveness of the cancer and can include any one or combination of the following procedures: surgical excision of the cancerous skin lesion to reduce the chance of recurrence and preserve healthy skin tissue; chemotherapy (e.g., dacarbazine, sorafnib), and radiation therapy. Additionally, even when widespread, melanoma can spontaneously regress. These rare instances seem to be related to a patient's developing immunity to the melanoma. Thus, much research in treatment of melanoma has focused on ways to get patients' immune system to react to their cancer, e.g., immunotherapy (e.g., Interleukin-2 (IL-2) and Interferon (IFN)), autologous vaccine therapy, adoptive T-Cell therapy, and gene therapy (used alone or in combination with surgicial procedures, chemotherapy, and/or radiation therapy).

Currently, the characterization of skin cancer, or conditions related to skin cancer is dependent on a person's ability to recognize the signs of skin cancer and perform regular self-examinations. An initial diagnosis is typically made from visual examination of the skin, a dermatoscopic exam, and patient feedback, and other questions about the patient's medical history. A definitive diagnosis of skin cancer and the stage of the disease's development can only be determined by a skin biopsy, i.e., removing a part of the lesion for microscopic examination of the cells, which causes the patient pain and discomfort. Metastatic melanomas can be detected by a variety of diagnostic procedures including X-rays, CT scans, MRIs, PET and PET/CTs, ultrasound, and LDH testing. However, once the cancer has metastasized, prognosis is very poor and can rapidly lead to death. Early detection of cancer, particularly melanoma, is crucial for a positive prognosis. Thus a need exists for better ways to diagnose and monitor the progression of skin cancer.

Additionally, information on any condition of a particular patient and a patient's response to types and dosages of therapeutic or nutritional agents has become an important issue in clinical medicine today not only from the aspect of efficiency of medical practice for the health care industry but for improved outcomes and benefits for the patients. Thus there is the need for tests which can monitor the progression and response to treatment, as well as predict the survival time of patients with melanoma.

### SUMMARY OF THE INVENTION

The invention is in based in part upon the identification of gene expression profiles (Precision Profiles™) associated with melanoma. These genes are referred to herein as melanoma genes or melanoma constituents. More specifically, the invention is based upon the surprising discovery that detection of as few as two melanoma survivability genes in a subject derived sample are capable of predicting the survivability and/or survival time of a patient suffering from melanoma. In addition, these genes are also predictive of a patients ability to respond to immunotherapy treatment, More particularly, the invention is based upon the surprising discovery that the methods provided by the invention are capable of predicting the response to immunotherapy, survivability and/or survival time of a melanoma-diagnosed subject by assaying blood samples. Even more surprisingly, the predictive nature of the genes shown in the Precision Profile™ for Melanoma (Table 1) is independent of any treatment of the melanoma diagnosed subject prior to blood draw.

The invention provides methods of evaluating the response to immunotherapy, survivability and/or survival time of a melanoma-diagnosed subject, based on a sample from the subject, the sample providing a source of RNAs, by determining a quantitative measure of the amount of at least one constituent of any constituent (e.g., melanoma gene) of Table 1, and arriving at a measure of each constituent, wherein such measure is obtained under measurement conditions that are substantially repeatable and the constituent is selected so that measurement of the constituent enables prediction of the response to immunotherapy, survivability or survival time of a melanoma-diagnosed subject. In particular embodiments, the invention provides methods of evaluating the predicted response to immunotherapy, survivability and/or survival time of a melanoma-diagnosed subject, based on the sample from the subject, the sample providing a source of RNAs, by determining a quantitative measure of a) at least two constituents according to any of the 2-gene models enumerated in Tables 3 and 9; b) at least three constituents according to any of the 2-gene models enumerated in Table 5; or c) at least four constituents according to any of the 4-gene models enumerated in Table 6; and arriving at a measure of each constituent, wherein such measure is obtained under measurement conditions that are substantially repeatable. In one preferred embodiment, at least four constituents are measured, wherein the four constituents are CTSD, PLA2G7 TXNRD1 and IRAK3. In another preferred embodiment the constituents that are measured are CTLA4 and ST14. In some embodiments CTLA4, ST14, IFI16 and ICAM ae measured. In another preferred embodiment the constituents that are measured are LARGE, NFKB1, BAX and TIMP1 and optionally one ore more constituents selected from RBM5, HMGA1 and HLADRA. Most preferably, LARGE, NFKB1, BAX, TIMP1, RBM5, HMGA1 and HLADRA.

In certain embodiments, the methods of the invention are capable of predicting survivability and/or survival time of a melanoma-diagnosed subject, wherein the subject is predicted to live 3 months, 6 months, 12 months, 1 year, 2, years, 3 years, 4 years, 5 years, 6 years, 7 years, 8 years, 9 years, 10 years, 15 years, 20 years, 30 years, 40 years, or 50 years from the date of diagnosis or date or initiating a therapeutic regimen for the treatment of melanoma.

Also provided are methods of assessing the effect of a particular variable, including but not limited to age, therapeutic agent, body mass index, ethnicity, and CTC count, on the predicted response to therapy, survivability and/or survival time of a subject based on a sample from the subject, the sample providing a source of RNAs, and determining a quantitative measure of the amount of at least one constituent of any constituent of Table 1 as a distinct RNA constituent in a sample obtained at a first period of time to produce a first subject data set and determining a quantitative measure of the amount of at least one constituent of any constituent of Table 1 as a distinct RNA constituent in a sample obtained at a second period of time (e.g., after administration of a therapeutic agent to said subject) to produce a second subject data set.

In a further aspect the invention provides methods of monitoring the progression of melanoma in a subject, based on a sample from the subject, the sample providing a source of RNAs, by determining a quantitative measure of the amount of at least one constituent of any constituent (e.g., melanoma survivability gene) of Table 1 as a distinct RNA constituent in a sample obtained at a first period of time to produce a first subject data set and determining a quantitative measure of the amount of at least one constituent of any constituent of Table 1 as a distinct RNA constituent in a sample obtained at a second period of time to produce a second subject data set. Optionally, the constituents measured in the first sample are the same constituents measured in the second sample. The first subject data set and the second subject data set are compared allowing effect of the agent on the predicted survivability and/or survival time to be determined. The second subject sample is taken e.g., one day, one week, one month, two months, three months, 1 year, 2 years, or more after the first subject sample. Optionally the first subject sample is taken prior to the subject receiving treatment, e.g. monoclonal antibody therapy, chemotherapy, radiation therapy, and/or surgery, and the second subject sample is taken after such treatment.

In various aspects the invention provides a method for determining a profile data set, i.e., a melanoma response to therapy profile, a melanoma survivability profile, for characterizing the predicted response to immunotherapy, survivability and/or survival time of a subject with melanoma based on a sample from the subject, the sample providing a source of RNAs and/or, by using amplification for measuring the amount of RNA in a panel of constituents including at least 1 constituent from Table 1, and arriving at a measure of each constituent. The profile data set contains the measure of each constituent of the panel.

In various aspects, the invention also provides a method for providing an index that is indicative of the predicted response to immunotherapy, survivability or survival time of a melanoma diagnosed subject, based on a sample from the subject, the method comprising: using amplification for measuring the amount of at least one constituent of Table 1 as a distinct RNA constituent in the subject sample, wherein such measure is obtained under measurement conditions that are substantially repeatable to form a first profile data set, and applying values from said first profile data set to an index function, thereby providing a single-valued measure of the predicted response to immunotherapy, probability of survivability or survival time so as to produce an index pertinent to the predicted survivability or survival time of the subject.

The methods of the invention further include comparing the quantitative measure of the constituent in the subject derived sample to a reference value. The reference value is for example an index value. Comparison of the subject measurements to a reference value allows for the the prediction of the primary endpoints of melanoma progression (e.g., metastasis, response to immunotherapy, and/or survivability) to be determined.

In various aspects of the invention the methods are carried out wherein the measurement conditions are substantially repeatable, particularly within a degree of repeatability of better than ten percent, five percent or more particularly within a degree of repeatability of better than three percent, and/or wherein efficiencies of amplification for all constituents are substantially similar, more particularly wherein the efficiency of amplification is within ten percent, more particularly wherein the efficiency of amplification for all constituents is within five percent, and still more particularly wherein the efficiency of amplification for all constituents is within three percent or less.

In addition, the one or more different subjects may have in common with the subject at least one of age group, gender, ethnicity, geographic location, nutritional history, medical condition, clinical indicator, medication, physical activity, body mass, and environmental exposure. A clinical indicator may be used to assess the predicted survivability and/or survival time of the one or more different subjects, and may also include interpreting the calibrated profile data set in the context of at least one other clinical indicator, wherein the at least one other clinical indicator includes blood chemistry, X-ray or other radiological or metabolic imaging technique, molecular markers in the blood (e.g., human leukocyte antigen (HLA) phenotype), other chemical assays, and physical findings.

At least 1, 2, 3, 4, 5, 6, 7, 8, 9, 10, 15, 20, 30 40, 50, 60, 70, 80, 90, 100, 110, 120, 130, 140, 150, 160 or more constituents are measured. The constituents are selected so as to predict the survivability and/or survival time of a melanoma-diagnosed subject with statistically significant accuracy. The melanoma-diagnosed subject is diagnosed with different stages of cancer. In one embodiment, the melanoma-diagnosed subject is advanced refractory and/or relapsed melanoma.

In one embodiment at least one constituent from Table 1 is measured. For example the at least one constituent measured is any of the constituents shown in Table 1 (i.e., the Precision Profile™ for Melanoma) or Table 2.

In another aspect, at least two constituents from Table 1 are measured. For example, two genes (i.e., constituents) according to any of the gene models listed in Table 3 or Table 9 are measured. Tables 3 and 9 describe examples of 2-gene models (e.g., CTSD and PLA2G7) derived from constituents listed in Table 1, capable of predicting the survivability of melanoma diagnosed subjects with highly statistically significant accuracy (p-value <0.05).

In yet another aspect, at least 3 constituents from Table 1 are measured. For example, 3 genes (i.e., constituents) according to any of the gene models listed in Table 5 are measured. Table 5 describes examples of 3-gene models (e.g., CTSD, PLA2G7 and TXNRD1) derived from constituents listed in Table 1, capable of predicting the survivability of melanoma diagnosed subjects with highly statistically significant accuracy (p-value <0.05).

In still another aspect, at least 4 constituents from Table 1 are measured. For example, 4-genes (i.e., constituents) according to any of the gene models listed in Table 6 are measured. Table 6 describes examples of 4-gene models (e.g., CTSD, PLA2G7, TXNRD1 and IRAK3) derived from constituents listed in Table 1, capable of predicting the survivability of melanoma diagnosed subjects with highly statistically significant accuracy (p-value <0.05).

Preferably, the constituents are selected so as to predict the survivability and/or survival time or a melanoma-diagnosed subject with at least 75%, 80%, 85%, 90%, 95%, 97%, 98%, 99% or greater accuracy. By “accuracy” is meant that the method has the ability to correctly predict theresponse to immunotherapy, survivability status and/or survival time of a melanoma diagnosed subject. Accuracy is determined for example by comparing the results of the Gene Precision Profiling™ to the survivability status of the subject (i.e., alive or dead).

In some embodiments, any of the models enumerated in any of Tables 2-3, 5-6 and 9 are combined (e.g., averaged) to form additional multi-gene models capable of predict the response to immunotherapy, survivability and/or survival time or a melanoma-diagnosed subject.

By melanoma or conditions related to melanoma is meant a type of skin cancer which develops from melanocytes, the skin cells in the epidermis which produce the skin pigment melanin. As used herein, melanoma includes melanoma, non-melanotic melanoma, nodular melanoma, acral lentiginous melanoma, and lentigo maligna.

The sample is any sample derived from a subject which contains RNA. For example, the sample is blood, blood fraction, body fluid, a population of cells or tissue from the subject, or a rare circulating tumor cell or circulating endothelial cell found in the blood.

Optionally one or more other samples can be taken over an interval of time between the first sample and the one or more other samples, or they may be taken pre-therapy intervention or post-therapy intervention. The therapy is for example, immunotherapy. In such embodiments, the first sample may be derived from blood and the baseline profile data set may be derived from tissue or body fluid of the subject other than blood. Alternatively, the first sample is derived from tissue or bodily fluid of the subject and the baseline profile data set is derived from blood.

Also included in the invention are kits for predicting response to therapy, the survivability and/or survival time of melanoma-diagnosed subject, containing at least one reagent for the detection or quantification of any constituent measured according to the methods of the invention and instructions for using the kit.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a graphical representation of low, medium and high risk groups established using the 4-gene model risk score, -2[(CTSD-TXNRD1)+(IRAK3-PLA2G7)], based on the Precision Profile™ for Melanoma Survivability (Table 1), capable of predicting the survivability of advanced refractory and/or relapsed melanoma. Subjects that fall above the upper diagonal line on the graph are in the low risk group, subjects that fall between the diagonal lines on the graph are in the medium risk group, and subjects that fall below the lower diagonal line are in the high risk group.

FIG. 2 is a cumulative survival curve (Kaplan Meier) based on the 4-gene Cox-type model, CTSD, PLA2G7, TXNRD1 and IRAK3.

FIG. 3 is a graphical representation of low, medium and high risk groups established using the 4-gene model risk score, -2[(CTSD-TXNRD1)+(IRAK3-PLA2G7)] to estimate the distribution of expected survival time by month for a latent class of subjects with advanced refractory melanoma predicted to survive >12 months. Subjects that fall above the upper line on the graph are in the low risk group (i.e., have a higher probability of surviving >12 months); subjects that fall between the lines on the graph are in the medium risk group, and subjects that fall below the line are in the high risk group (i.e., have a lower probability of serving >12 months).

FIG. 4 is a cumulative survival curve (Meier Kaplan) based on the expected frequencies from two latent classes identified using the 4-gene Cox-type model, CTSD, PLA2G7, TXNRD1 and IRAK3.

FIG. 5 shows a survival curve (Kaplan Meier) based on the 2-gene Cox-type model, CTLA4 and ST14).

FIG. 6 shows a survival curve (Kaplan Meier) based on the 2-gene Cox-type model, CTLA4 and ST14 using prespecified percentile groups.

FIG. 7 shows a survival curve (Kaplan Meier) based on the 4-gene Cox-type model, CTLA4, ST14, IF116 and ICAM1 using the pre-specified risk score

FIG. 8 shows a survival curve (Kaplan Meier) based on the n the 4-gene Cox-type CTLA4, ST14, IF116 and ICAM1 using prespecified percentile groups.

FIG. 9 shows a receiver operator curves (ROC) based on the 1008 population.

FIG. 10 shows that the seven gene K component model distinguishes prime and proxy genes.

FIG. 11 shows that the seven gene K-component model distinguishes subjects who will respond to immunotherapy to those that will not.

FIG. 12 shows that similar results are obtained using a logistic regression model based upon the seven gen K-component model.

FIG. 13 shows receiver operator curves (ROC) comparing the 7 gene K-component model and the logistic regression model.

FIG. 14 shows the ability of the 7 gene K-component model to select subjects who will respond to immunotherapy compared to traditional CRP measurements

FIG. 15 shows survival curves (Kaplan Meier) demonstrating that the seven gene K-component model also is highly predictive of survival.

### DETAILED DESCRIPTION

### Definitions

The following terms shall have the meanings indicated unless the context otherwise requires:
“Accuracy” refers to the degree of conformity of a measured or calculated quantity (a test reported value) to its actual (or true) value. Clinical accuracy relates to the proportion of true outcomes (true positives (TP) or true negatives (TN)) versus misclassified outcomes (false positives (FP) or false negatives (FN)), and may be stated as a sensitivity, specificity, positive predictive values (PPV) or negative predictive values (NPV), or as a likelihood, odds ratio, among other measures.
“Algorithm” is a set of rules for describing a biological condition. The rule set may be defined exclusively algebraically but may also include alternative or multiple decision points requiring domain-specific knowledge, expert interpretation or other clinical indicators.
An “agent” is a “composition” or a “stimulus”, as those terms are defined herein, or a combination of a composition and a stimulus.
“Amplification” in the context of a quantitative RT-PCR assay is a function of the number of DNA replications that are required to provide a quantitative determination of its concentration.
A “baseline data set” is a set of values associated with an indicator resulting from evaluation of a biological sample (or population or set of samples) under a desired biological condition that is used for mathematically normative purposes. The desired biological condition may be, for example, the condition of a subject (or population or set of subjects) before exposure to an agent or in the presence of an untreated disease or in the absence of a disease. Alternatively, or in addition, the desired biological condition may be health of a subject or a population or set of subjects. Alternatively, or in addition, the desired biological condition may be that associated with a population or set of subjects selected on the basis of at least one of age group, gender, ethnicity, geographic location, nutritional history, medical condition, clinical indicator, medication, physical activity, body mass, and environmental exposure.
A “biological state” of a subject is the condition of the subject, as with respect to circumstances or attributes of the biological condition.
A “biological condition” of a subject is the condition of the subject in a pertinent realm that is under observation, and such realm may include any aspect of the subject capable of being monitored for change in condition, such as health; disease including cancer; trauma; aging; infection; tissue degeneration; developmental steps; physical fitness; obesity; and mental state. As can be seen, a condition in this context may be chronic or acute or simply transient. Moreover, a targeted biological condition may be manifest throughout the organism or population of cells or may be restricted to a specific organ (such as skin, heart, eye or blood) but in either case, the condition may be monitored directly by a sample of the affected population of cells or indirectly by a sample derived elsewhere from the subject. The term “biological condition” includes a “physiological condition”. For example, the biological condition is cancer such as prostate cancer, ovarian cancer, lung cancer, breast cancer, skin cancer, colon cancer, or cervical cancer.
“Biomarker(s)” can be classified based on different parameters. They can be classified based on their characteristics such as imaging biomarkers (CT, PET, MRI) or molecular biomarkers. Molecular biomarkers can be used to refer to nonimaging biomarkers that have biophysical properties, which allow their measurements in biological samples (eg, plasma, serum, cerebrospinal fluid, bronchoalveolar lavage, biopsy) and include nucleic acids-based biomarkers such as gene mutations or polymorphisms and quantitative gene expression analysis, peptides, proteins, lipids metabolites, and other small molecules. Biomarkers can also be classified based on their application such as diagnostic biomarkers, staging of disease biomarkers, disease prognosis biomarkers, and biomarkers for monitoring the clinical response to an intervention. Another category of biomarkers includes those used in decision making in early drug development. For instance, pharmacodynamic (PD) biomarkers are markers of a certain pharmacological response, which are of special interest in dose optimization studies.
“Body fluid” of a subject includes blood, urine, spinal fluid, lymph, mucosal secretions, prostatic fluid, semen, haemolymph or any other body fluid known in the art for a subject.
“Calibrated data set” is a function of a member of a first data set and a corresponding member of a baseline data set for a given constituent in a panel.
A “circulating endothelial cell” (“CEC”) is an endothelial cell from the inner wall of blood vessels which sheds into the bloodstream under certain circumstances, including inflammation, and contributes to the formation of new vasculature associated with cancer pathogenesis. CECs may be useful as a marker of tumor progression and/or response to antiangiogenic therapy.
A “circulating tumor cell” (“CTC”) is a tumor cell of epithelial origin which is shed from the primary tumor upon metastasis, and enters the circulation. The number of circulating tumor cells in peripheral blood is associated with prognosis in patients with metastatic cancer. These cells can be separated and quantified using immunologic methods that detect epithelial cells.
A “clinical indicator” is any physiological datum used alone or in conjunction with other data in evaluating the physiological condition of a collection of cells or of an organism. This term includes pre-clinical indicators.
“Clinical parameters” encompasses of a subject's health status or other characteristics, such as, without limitation, age (AGE), ethnicity (RACE), gender (SEX), and family history of disease, such as cancer. A clinical parameter is also referred to as a covariate.
A “Composition” includes a chemical compound, a nutraceutical, a pharmaceutical, a homeopathic formulation, an allopathic formulation, a naturopathic formulation, a combination of compounds, a toxin, a food, a food supplement, a mineral, and a complex mixture of substances, in any physical state or in a combination of physical states.
A “Control Value” is a value obtained from a reference sample(s) in which the biological state is known. The control value may be an index.
“Correlation Coefficient” is a measure of the interdependence of two random variables that ranges in value from −1 to +1, indicating perfect negative correlation at −1, absence of correlation at zero, and perfect positive correlation at +1. Also called coefficient of correlation. There are several correlation coefficients, often denoted ρ or r, measuring the degree of correlation. The most common of these is the Pearson correlation coefficient, which is mainly sensitive to a linear relationship between two variables. Other correlation coefficients have been developed to be more robust than the Pearson correlation, or more sensitive to nonlinear relationships The most familiar measure of dependence between two quantities is the Pearson product-moment correlation coefficient, or “Pearson's correlation.” It is obtained by dividing the covariance of the two variables by the product of their standard deviations.
“Correlated” is meant that that correlation coefficient is greater than 0.1; 0.2; 0.3; 0.4; 0.5; 0.6; 0.7; 0.8; or 0.9. Preferably, the correlation coefficient is great at least 0.5 or greater.
To “derive” a data set from a sample includes determining a set of values associated with the indicator either (i) by direct measurement of such indicator in a biological sample or (ii) by indirect measurement of such indicator in a biological sample.
A “Digital computer system” includes a programmable calculator or other programmable device.
“Distinct RNA or protein constituent” is a distinct expressed product of a gene, whether RNA or protein. An “expression” product of a gene includes the gene product whether RNA or protein resulting from translation of the messenger RNA.
“Enumerated or Enumeration” is meant to to ascertain the number of possible models predicative of a biological state. See, for example the enumeration methodology described in Example 2.
“FN” is false negative, which for a disease state test means classifying a disease subject incorrectly as non-disease or normal.
“FP” is false positive, which for a disease state test means classifying a normal subject incorrectly as having disease.
A “formula,” “algorithm,” or “model” is any mathematical equation, algorithmic, analytical or programmed process, statistical technique, or comparison, that takes one or more continuous or categorical inputs and calculates an output value, sometimes referred to as an “index” or “index value.” Non-limiting examples of “formulas” include comparisons to reference values or profiles, sums, ratios, and regression operators, such as coefficients or exponents, value transformations and normalizations (including, without limitation, those normalization schemes based on clinical parameters, such as gender, age, or ethnicity), rules and guidelines, statistical classification models, and neural networks trained on historical populations. Of particular use in combining indicators are linear and non-linear equations and statistical significance and classification analyses to determine the relationship between levels of a indicator detected in a subject sample and the survivability of the subject. Techniques which may be used in survival and time to event hazard analysis, include but are not limited to Cox, Zero-Inflation Poisson, Markov, Weibull, Kaplan-Meier and Greenwood models, well known to those of skill in the art. In panel and combination construction, of particular interest are structural and synactic statistical classification algorithms, and methods of risk index construction, utilizing pattern recognition features, including, without limitation, such established techniques such as cross-correlation, Principal Components Analysis (PCA), factor rotation, Logistic Regression Analysis (LogReg), Kolmogorov Smirnoff tests (KS), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques (CART, LART, LARTree, FlexTree, amongst others), Shrunken Centroids (SC), StepAIC, K-means, Kth-Nearest Neighbor, Boosting, Decision Trees, Neural Networks, Bayesian Networks, Support Vector Machines, and Hidden Markov Models, among others. Many of these techniques are useful either combined with a an indicator selection technique, such as forward selection, backwards selection, or stepwise selection, complete enumeration of all potential panels of a given size, genetic algorithms, voting and committee methods, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other clinical studies, or cross-validated within the study they were originally trained in, using such techniques as Bootstrap, Leave-One-Out (LOO) and 10-Fold cross-validation (10-Fold CV). At various steps, false discovery rates (FDR) may be estimated by value permutation according to techniques known in the art. A “Gene Expression Panel” (Precision Profile™) is an experimentally verified set of constituents, each constituent being a distinct expressed product of a gene, whether RNA or protein, wherein constituents of the set are selected so that their measurement provides a measurement of a targeted biological condition.
A “Gene Expression Profile” is a set of values associated with constituents of a Gene Expression Panel (Precision Profile™) resulting from evaluation of a biological sample (or population or set of samples).
A “Gene Expression Profile Inflammation Index” is the value of an index function that provides a mapping from an instance of a Gene Expression Profile into a single-valued measure of inflammatory condition.
A Gene Expression Profile Cancer Index” is the value of an index function that provides a mapping from an instance of a Gene Expression Profile into a single-valued measure of a cancerous condition.
The “health” of a subject includes mental, emotional, physical, spiritual, allopathic, naturopathic and homeopathic condition of the subject.
“Index” is an arithmetically or mathematically derived numerical characteristic developed for aid in simplifying or disclosing or informing the analysis of more complex quantitative information. A survivability and/or survival time index may be determined by the application of a specific algorithm to a plurality of subjects or samples with a common biological condition.
“Indicator” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Indicator can also include mutated proteins or mutated nucleic acids. Indicator also encompass non-blood borne factors or non-analyte physiological markers of health status, such as “clinical parameters” defined herein, as well as “traditional laboratory risk factors”, also defined herein. Indicators also include any calculated indices created mathematically or combinations of any one or more of the foregoing measurements, including temporal trends and differences. Where available, and unless otherwise described herein, biomarkers which are gene products are identified based on the official letter abbreviation or gene symbol assigned by the international Human Genome Organization Naming Committee (HGNC) and listed at the date of this filing at the US National Center for Biotechnology Information (NCBI) web site (http://www.ncbi.nlm.nih.gov/sites/entrez?db=gene), also known as Entrez Gene. An indicator is for example a biomarker.
“Inflammation” is used herein in the general medical sense of the word and may be an acute or chronic; simple or suppurative; localized or disseminated; cellular and tissue response initiated or sustained by any number of chemical, physical or biological agents or combination of agents.
“Inflammatory state” is used to indicate the relative biological condition of a subject resulting from inflammation, or characterizing the degree of inflammation.
A “large number” of data sets based on a common panel of genes is a number of data sets sufficiently large to permit a statistically significant conclusion to be drawn with respect to an instance of a data set based on the same panel.
“Measuring” or “measurement,” means assessing the presence, absence, quantity or amount of either a given substance within a clinical or subject-derived sample, including the derivation of qualitative, semi-quantitative or quantitative concentration levels of such substances, or otherwise evaluating the values or categorization of a subject's non-analyte clinical parameters.
“Melanoma” is a type of skin cancer which develops from melanocytes, the skin cells in the epidermis which produce the skin pigment melanin. As used herein, melanoma includes Stage I, Stage II, Stage III and Stage IV melanoma, as determined by the AJCC (6^{th} Edition), non-melanotic melanoma, nodular melanoma, acral lentiginous melanoma, and lentigo maligna. “Active melanoma” indicates a subject having melanoma with clinical evidence of disease, and includes subjects that have had blood drawn within 2-3 weeks post resection, although no clinical evidence of disease may be present after resection. “Inactive melanoma” indicates subjects having no clinicial evidence of disease.
“Non-melanoma” is a type of skin cancer which develops from skin cells other than melanocytes, and includes basal cell carcinoma, squamous cell carcinoma, cutaneous T-cell lymphoma, Merkel cell carcinoma, dermatofibrosarcoma protuberans, and Paget's disease.
“Molecular risk assessment” means a procedure in which biomarkers (i.e., indicators) are used to estimate a person's risk for developing a biological condition
“Negative predictive value” or “NPV” is calculated by TN/(TN+FN) or the true negative fraction of all negative test results. It also is inherently impacted by the prevalence of the disease and pre-test probability of the population intended to be tested.
See, e.g., O'Marcaigh A S, Jacobson R M, “Estimating the Predictive Value of a Diagnostic Test, How to Prevent Misleading or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al., “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation of Laboratory Procedures,” chapter 14 in Teitz, Fundamentals of Clinical Chemistry, Burtis and Ashwood (eds.), 4^{th} edition 1996, W. B. Saunders Company, pages 192-199; and Zweig et al., “ROC Curve Analysis: An Example Showing the Relationships Among Serum Lipid and Apolipoprotein Concentrations in Identifying Subjects with Coronory Artery Disease,” Clin. Chem., 1992, 38(8): 1425-1428. An alternative approach using likelihood functions, BIC, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.
A “normal” subject is a subject who is generally in good health, has not been diagnosed with a biological condition, e.g., is asymptomatic for prostate cancer, and lacks the traditional laboratory risk factors for the biological condition.
A “normative value” is the value of the indicator in a normal subject.
An “Outcome category”, synonymous with “outcome” refers to a particular category of a “categorical outcome variable”
An “Outcome score”, synonymous with “outcome value”, refers to a quantitative value associated with a given category or level of an ‘outcome variable’.
An “Outcome variable” is a variable containing at least one set of scores that are believed to be correlated with an underlying biological condition of the cases, and may be categorical (“categorical outcome variable”) which may be nominal or ordinal, continuous or may denote an event history.
A “Panel” is an experimentally verified set of indicators. A “panel” includes a set of at least two indicators.
A “Profile” is a set of values associated with constituents of an indicator resulting from evaluation of a biological sample (or population or set of samples).
A “population of cells” refers to any group of cells wherein there is an underlying commonality or relationship between the members in the population of cells, including a group of cells taken from an organism or from a culture of cells or from a biopsy, for example.
“Positive predictive value” or “PPV” is calculated by TP/(TP+FP) or the true positive fraction of all positive test results. It is inherently impacted by the prevalence of the disease and pre-test probability of the population intended to be tested.
“Prime indicator” is an indicator that alone makes a statistically significant contribution to the evaluation of the biological state. Optimally, the change in the value of the prime indicator in a normal subject compared to a subject with an altered biological is greater than the standard of error of the test that is used to measure the value.
“Proxy indicator” is an indicator that alone does not make a statistically significant contribution to the evaluation of the biological state, is correlated with the prime indicator and whose value is similar in both a normal biological state and an altered biological state.
“Risk” in the context of the present invention, relates to the probability that an event will occur over a specific time period, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of lower risk cohorts, across population divisions (such as tertiles, quartiles, quintiles, or deciles, etc.) or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no-conversion.
“Risk evaluation,” or “evaluation of risk” in the context of the present invention encompasses making a prediction of the probability, odds, or likelihood that an event (e.g., death) or disease state may occur, and/or the rate of occurrence of the event (e.g., death) or conversion from one disease state to another, i.e., from a normal condition to cancer or from cancer remission to cancer, or from primary cancer occurrence to occurrence of a cancer metastasis. Risk evaluation can also comprise prediction of future clinical parameters, traditional laboratory risk factor values, or other indices of cancer results, either in absolute or relative terms in reference to a previously measured population. Such differing use may require different combinations and individualized panels, mathematical algorithms, and/or cut-off points, but be subject to the same aforementioned measurements of accuracy and performance for the respective intended use.
A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art. The sample is blood, urine, spinal fluid, lymph, mucosal secretions, prostatic fluid, semen, haemolymph or any other body fluid known in the art for a subject. The sample is also a tissue sample. The sample is or contains a circulating endothelial cell or a circulating tumor cell.
“Sensitivity” is calculated by TP/(TP+FN) or the true positive fraction of disease subjects.
“Specificity” is calculated by TN/(TN+FP) or the true negative fraction of non-disease or normal subjects.
By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less and statistically significant at a p-value of 0.10 or less. Such p-values depend significantly on the power of the study performed. By non-statistically significant it is mean a p-value greater than 0.05.
A “set” or “population” of samples or subjects refers to a defined or selected group of samples or subjects wherein there is an underlying commonality or relationship between the members included in the set or population of samples or subjects.
A “subject” is a cell, tissue, or organism, human or non-human, whether in vivo, ex vivo or in vitro, under observation. As used herein, reference to predicting the survivability and/or survival time of a subject based on a sample from the subject, includes using blood or other tissue sample from a human subject to evaluate the human subject's predicted survivability and/or survival time; it also includes, for example, using a blood sample itself as the subject to evaluate, for example, the effect of therapy or an agent upon the sample.
A “stimulus” includes (i) a monitored physical interaction with a subject, for example ultraviolet A or B, or light therapy for seasonal affective disorder, or treatment of psoriasis with psoralen or treatment of cancer with embedded radioactive seeds, other radiation exposure, and (ii) any monitored physical, mental, emotional, or spiritual activity or inactivity of a subject.
“Survivability” refers to the ability to remain alive or continue to exist (i.e., alive or dead).
“Survival time” refers to the length or period of time a subject is able to remain alive or continue to exist as measured from an initial date (e.g., date of birth, date of diagnosis of a particular disease or stage of disease, date of initiating a therapeutic regimen, etc.) to a later date in time (e.g., date of death, date of termination of a particular therapeutic regimen, or an arbitrary date).
“Therapy” or “therapeutic regimen” includes all interventions whether biological, chemical, physical, metaphysical, or combination of the foregoing, intended to sustain or alter the monitored biological condition of a subject.
“TN” is true negative, which for a disease state test means classifying a non-disease or normal subject correctly.
“TP” is true positive, which for a disease state test means correctly classifying a disease subject.
A “value” is a numerical quantity measured, assigned or computed for the indicator.

The PCT patent application publication number WO 01/25473, published Apr. 12, 2001, entitled “Systems and Methods for Characterizing a Biological Condition or Agent Using Calibrated Gene Expression Profiles,” which is herein incorporated by reference, discloses the use of Gene Expression Panels (Precision Profiles™) for the evaluation of (i) biological condition (including with respect to health and disease) and (ii) the effect of one or more agents on biological condition (including with respect to health, toxicity, therapeutic treatment and drug interaction). The PCT patent application PCT/US2007/023425, filed Nov. 6, 2007, entitled “Gene Expression Profᵢling for Identification, Monitoring and Treatment of Melanoma”, filed for an invention by the inventors herein, and which is herein incorporated by reference in its entirety, discloses the use of Gene Expression Panels (Precision Profiles™) for evaluating the presence or likelihood of melanoma in a subject, and for monitoring response to therapy in a melanoma-diagnosed subject, and for monitoring the progression of melanoma in a melanoma-diagnosed subject (i.e., cancer versus a normal, healthy, disease free state).

The present invention provides a Gene Expression Panel (Precision Profile™) for predicting the response to immunotherapy, survivability and/or survival time of a melanoma-diagnosed subject and for evaluating the effect of one or more variables on the predicted response to immunotherapy, survivability and/or survival time of a melanoma-diagnosed subject. The Gene Expression Panel (Precision Profile™) described herein may be used for identifying and assessing predictive relationships between RNA-transcript-based gene expression and predicted response to immunotherapy, survivability and/or survival time of a melanoma diagnosed subject (either direct relationship or indirect relationship, e.g., affecting the latent classes). For example, the Gene Expression Panel (Precision Profile™) described herein may be used, without limitation, for measurement of the following with respect to a melanoma-diagnosed subject: response to immunotherapy, predicting the survivability, predicting the expected survival time, predicting the probability of long-term survivability, predicting the effect of one or more variables. (including without limitation, age, therapeutic regimen, body mass index, ethnicity, family history of cancer) on survivability and/or survival time, and for predicting the survivability and/or survival time of latent classes (e.g., distinguishing the predicted survivability and/or survival times of a set or population of melanoma-diagnosed subjects having the same or different clinical presentation (e.g., tumor volume, tumor location, stage of disease, etc.)). Survivability and/or survival time can be predicted within 3 months, 6 months, 1 years, 2, years, 3, years, 4 years, 5 years, 6 years, 7 years, 8 years, 9 years, 10 years, 15 years, 20 years 30 years 40 years or 50 years within the date of diagnosis or date of initiating a therapeutic regimen for the treatment of melanoma.

The Gene Expression Panel (Precision Profile™) may be employed with respect to samples derived from subjects in order to evaluate their predicted response to immunotherapy, survivability and/or survival time. The Gene Expression Panel (Precision Profile™) is referred to herein as the Precision Profile™ for Melanoma (Table 1), which includes one or more genes, e.g., constituents, whose expression is associated with inflammation, melanoma, and the CTLA4 pathway. Each gene of the Precision Profile™ for Melanoma is referred to herein as a melanoma gene or a melanoma constituent.

It has been discovered that valuable and unexpected results may be achieved when the quantitative measurement of constituents is performed under repeatable conditions (within a degree of repeatability of measurement of better than twenty percent, preferably ten percent or better, more preferably five percent or better, and more preferably three percent or better). For the purposes of this description and the following claims, a degree of repeatability of measurement of better than twenty percent may be used as providing measurement conditions that are “substantially repeatable”. In particular, it is desirable that each time a measurement is obtained corresponding to the level of expression of a constituent in a particular sample, substantially the same measurement should result for substantially the same level of expression. In this manner, expression levels for a constituent in a Gene Expression Panel (Precision Profile™) may be meaningfully compared from sample to sample. Even if the expression level measurements for a particular constituent are inaccurate (for example, say, 30% too low), the criterion of repeatability means that all measurements for this constituent, if skewed, will nevertheless be skewed systematically, and therefore measurements of expression level of the constituent may be compared meaningfully. In this fashion valuable information may be obtained and compared concerning expression of the constituent under varied circumstances.

In addition to the criterion of repeatability, it is desirable that a second criterion also be satisfied, namely that quantitative measurement of constituents is performed under conditions wherein efficiencies of amplification for all constituents are substantially similar as defined herein. When both of these criteria are satisfied, then measurement of the expression level of one constituent may be meaningfully compared with measurement of the expression level of another constituent in a given sample and from sample to sample.

The prediction of the rsurvivability of a melanoma-diagnosed subject is defined to be a prediction of the survivability and/or survival time of the subject and/or the assessment of the effect of a particular variable (e.g., age, therapeutic agent, body mass index, ethnicity, CTC count) on the predicted survivability and/or survival time.

The agent to be evaluated for its effect on the survivability of a melanoma-diagnosed subject may be a compound known to treat melanoma or compounds that are not known to treat melanoma. Compounds for the treatment of melanoma are well known in the art and include but are not limited to various forms of chemotherapy, immunotherapy, monoclonal antibody therapy, gene therapy, adoptive T-cell therapy, and vaccine therapy.

The predicted response to immunotherapy, survivability and/or survival time of a melanoma-diagnosed subject is evaluated by determining the level of expression (e.g., a quantitative measure) of an effective number (e.g., one or more) of constituents of the Precision Profile™ for Melanoma (Table 1). By an effective number is meant the number of constituents that need to be measured in order to directly predict response to immunotherapy, the survivability and/or survival time of a melanoma-diagnosed subject, and/or to predict the survivability and/or survival time of latent classes (e.g., melanoma subject having the same or different clinical presentation). Preferably the constituents are selected as to predict the response to immunotherapy, survivability and/or survival time of a melanoma-diagnosed subject with least 75% accuracy, more preferably 80%, 85%, 90%, 95%, 97%, 98%, 99% or greater accuracy.

The level of expression is determined by any means known in the art. For example, the level of expression of one or more constituents of the Precision Profile™ for Melanoma (Table 1) is measured by quantitative PCR. The measurement is obtained under conditions that are substantially repeatable. Optionally, the qualitative measure of the constituent is compared to a reference or baseline level or value (e.g. a baseline profile set). In one embodiment, the reference or baseline level is the predicted response to immunotherapy, survivability and/or survival time as a function of variable subject factors such as age, metastatic status and/or treatment, without the use of constituent measurements. In another embodiment, the reference or baseline level is derived from the same subject from which the first measure is derived. For example, the baseline is taken from a subject at different time periods, (e.g., prior to receiving treatment or surgery for melanoma, or at different time periods during a course of treatment). Such methods allow for the evaluation of the effect of a particular variable (e.g., treatment for a selected individual) on the survivability of a melanoma diagnosed subject. Such methods also allow for the evaluation of the effect of a particular variable (e.g., treatment) on the expression levels of one or more constituents which are capable of predicting the survivability of a melanoma diagnosed subject. Comparison can be performed on test (e.g., patient) and reference samples (e.g., baseline) measured concurrently or at temporally distinct times. An example of the latter is the use of compiled expression information, e.g., a gene expression database, which assembles information about expression levels of cancer associated genes.

A reference or baseline level or value as used herein can be used interchangeably and is meant to be relative to a number or value derived from population studies, including without limitation, such subjects having similar age range, disease status (e.g., stage), subjects in the same or similar ethnic group, or relative to the starting sample of a subject undergoing treatment for melanoma. Such reference values can be derived from statistical analyses and/or risk prediction data of populations obtained from mathematical algorithms and computed indices of melanoma. Reference indices can also be constructed and used using algorithms and other methods of statistical and structural classification.

In one embodiment of the present invention, the reference or baseline value is the amount of expression of a cancer associated gene in a control sample derived from one or more melanoma-diagnosed subjects who have not received any treatment for melanoma.

In another embodiment of the present invention, the reference or baseline value is the level of cancer survivability associated genes in a control sample derived from one or more melanoma diagnosed subjects who have received a therapeutic regimen to treat melanoma.

In a further embodiment, such subjects are monitored and/or periodically retested for a diagnostically relevant period of time (“longitudinal studies”) following such test to verify continued response to immunotherapy, survivability, or lack thereof. Such period of time may be one year, two years, two to five years, five years, five to ten years, ten years, or ten or more years from the initial testing date for determination of the reference or baseline value. Furthermore, retrospective measurement of cancer survivability associated genes in properly banked historical subject samples may be used in establishing these reference or baseline values, thus shortening the study time required, presuming the subjects have been appropriately followed during the intervening period through the intended horizon of the product claim.

A reference or baseline value can also comprise the amounts of cancer survivability associated genes derived from subjects who show an improvement in cancer status as a result of treatments and/or therapies for the cancer being treated and/or evaluated.

For example, where the reference or baseline level is comprised of the amounts of cancer survivability associated genes derived from one or more melanoma diagnosed subjects who have not received any treatment for melanoma, a change (e.g., increase or decrease) in the expression level of a cancer survivability associated gene in the patient-derived sample as compared to the expression level of such gene in the reference or baseline level indicates that the particular therapeutic may have an effect on the predicted survivability and/or survival time of the subject.

Expression of a melanoma gene also allows for the course of treatment of melanoma to be monitored and evaluated for an effect on the predicted response to immunotherapy, survivability and/or survival time of a melanoma-diagnosed subject. In this method, a biological sample is provided from a subject undergoing treatment, e.g., if desired, biological samples are obtained from the subject at various time points before, during, or after treatment. Expression of a melanoma survivability gene is then determined and compared to a reference or baseline profile. The baseline profile may be taken or derived from one or more individuals who have been exposed to the treatment. Alternatively, the baseline level may be taken or derived from one or more individuals who have not been exposed to the treatment. For example, samples may be collected from subjects who have received initial treatment for melanoma and subsequent treatment for melanoma to monitor whether the course of treatment has an affect on the predicted survivability and/or survival time of the subject.

A Gene Expression Panel (Precision Profile™) is selected in a manner so that quantitative measurement of RNA or protein constituents in the Panel constitutes a measurement of the predicted survivability and/or survival time of a subject. In one kind of arrangement, a calibrated profile data set is employed. Each member of the calibrated profile data set is a function of (i) a measure of a distinct constituent of a Gene Expression Panel (Precision Profile™) and (ii) a baseline quantity.

Additional embodiments relate to the use of an index or algorithm resulting from quantitative measurement of constituents, and optionally in addition, derived from either expert analysis or computational biology, useful as a prognostic tool for predicting the response to immunotherapy, survivability and/or survival times of a melanoma-diagnosed subject (e.g., as a direct effect or affecting latent classes).

Gene expression profiling and the use of index characterization for a particular condition or agent or both may be used to reduce the cost of Phase 3 clinical trials and may be used beyond Phase 3 trials; labeling for approved drugs; selection of suitable medication in a class of medications for a particular patient that is directed to their unique physiology; diagnosing or determining a prognosis of a medical condition or an infection which may precede onset of symptoms or alternatively diagnosing adverse side effects associated with administration of a therapeutic agent; and managing the health care of a patient.

### The subject

The methods disclosed herein may be applied to cells of humans, mammals or other organisms without the need for undue experimentation by one of ordinary skill in the art because all cells transcribe RNA and it is known in the art how to extract RNA from all types of cells.

A subject can include those who have already been diagnosed as having skin cancer or a condition related to skin cancer (e.g., melanoma). Diagnosis of skin cancer is made, for example, from any one or combination of the following procedures: a medical history; a visual examination of the skin looking for common features of cancerous skin lesions, including but not limited to bumps, shiny translucent, pearly, or red nodules, a sore that continuously heals and re-opens, a crusted or scaly area of the skin with a red inflamed base that resembles a growing tumor, a non-healing ulcer, crusted-over patch of skin, new moles, changes in the size, shape, or color of an existing mole, the spread of pigmentation beyond the border of a mole or mark, oozing or bleeding from a mole, and a mole that feels itchy, hard, lumpy, swollen, or tender to the touch; a dermatoscopic exam; imaging techniques including X-rays, CT scans, MRIs, PET and PET/CTs, ultrasound, and LDH testing; and biopsy, including shave, punch, incisional, and excsisional biopsy.

A subject can also include those who are suffering from different stages of skin cancer, e.g., Stage 1 through Stage 4 melanoma. An individual diagnosed with Stage 1 indicates that no lymph nodes or lymph ducts contain cancer cells (i.e., there are no positive lymph nodes) and there is no sign of cancer spread. In this stage, the primary melanoma is less than 2.0 mm thick or less than 1.0 mm thick and ulcerated, i.e., the covering layer of the skin over the tumor is broken. Stage 2 melanomas also have no sign of spread or positive lymph nodes Stage 2 melanomas are over 2.0 mm thick or over 1.0 mm thick and ulcerated. Stage 3 indicates all melanomas where there are positive lymph nodes, but no sign of the cancer having spread anywhere else in the body. Stage 4 melanomas have spread elsewhere in the body, away from the primary site.

A subject can also include those who are suffering from, or at risk of developing skin cancer or a condition related to skin cancer (e.g., melanoma), such as those who exhibit known risk factors skin cancer. Known risk factors for skin cancer include, but are not limited to cumulative sun exposure, blond or red hair, blue eyes, fair complexion, many freckles, severe sunburns as a child, family history of skin cancer (e.g., melanoma), dysplastic nevi, atypical moles, multiple ordinary moles (>50), immune suppression, age, gender (increased frequency in men), xeroderma pigmentosum (a rare inherited condition resulting in a defect from an enzyme that repairs damage to DNA), and past history of skin cancer.

Optionally, the subject has been previously treated with a surgical procedure for removing skin cancer or a condition related to skin cancer (e.g., melanoma), including but not limited to any one or combination of the following treatments: cryosurgery, i.e., the process of freezing with liquid nitrogen; curettage and electrodessication, i.e., the scraping of the lesion and destruction of any remaining malignant cells with an electric current; removal of a lesion layer-by-layer down to normal margins (Moh's surgery).

Optionally, the subject has previously been treated with any one or combination of therapeutic treatments for melanoma, alone, or in combination with a surgical procedure for removing skin cancer. Therapeutic treatments for melanoma are known in the art and include but are not limited to chemotherapy, immunotherapy, monoclonal antibody therapy, gene therapy, adoptive T-cell therapy, and vaccine therapy.

### Selecting Constituents of a Gene Expression Panel (Precision Profile™)

The general approach to selecting constituents of a Gene Expression Panel (Precision Profile™) has been described in PCT application publication number WO 01/25473, incorporated herein in its entirety. A wide range of Gene Expression Panels (Precision Profiles™) have been designed and experimentally validated, each panel providing a quantitative measure of biological condition that is derived from a sample of blood or other tissue. For each panel, experiments have verified that a Gene Expression Profile using the panel's constituents is informative of a biological condition (it has also been demonstrated that in being informative of biological condition, the Gene Expression Profile is used, among other things, to measure the effectiveness of therapy, as well as to provide a target for therapeutic intervention).

### Gene Expression Profiles Based on Gene Expression Panels (Precision Profiles™) of the Present Invention

Tables 2-3, 5-6 and 9 were derived from a study of the gene expression patterns in subjects with advanced refractory and/or relapsed melanoma, based on the Precision Profile™ for Melanoma (Table 1), as described in Example 1 below.

Table 2 describes all statistically significant 1-gene models based on genes from the Precision Profile™ for Melanoma (Table 1) which were identified by using a Cox-type survival model as capable of predicting the survivability of a subject with advanced refractory and/or relapsed melanoma.

Table 3 describe examples of statistically significant 2-gene models based on genes from the Precision Profile™ for Melanoma (Table 1) which were identified using a Cox-type survival model as capable of predicting survivability of a subject with advanced refractory and/or relapsed melanoma.

Table 5 describes examples of statistically significant 3 gene models identified by using a Cox-type survival model capable of predicting the survivability of a subject with advanced refractory and/or relapsed melanoma.

Table 6 describes examples of statistically significant 4-gene models identified by using a Cox-type survival model, capable of predicting the survivability of a subject with advanced refractory and/or relapsed melanoma.

Table 9 describes additional examples of statistically significant 2-gene models based on genes from the Precision Profile™ for Melanoma (Table 1) which were identified using a Cox-type survival model as capable of predicting survivability of a subject with advanced refractory and/or relapsed melanoma.

### Design of Assays

Typically, a sample is run through a panel in replicates of three for each target gene (assay); that is, a sample is divided into aliquots and for each aliquot the concentrations of each constituent in a Gene Expression Panel (Precision Profile™) is measured. From over thousands of constituent assays, with each assay conducted in triplicate, an average coefficient of variation was found (standard deviation/average)*100, of less than 2 percent among the normalized ΔCt measurements for each assay (where normalized quantitation of the target mRNA is determined by the difference in threshold cycles between the internal control (e.g., an endogenous marker such as 18S rRNA, or an exogenous marker) and the gene of interest. This is a measure called “intra-assay variability”. Assays have also been conducted on different occasions using the same sample material. This is a measure of “inter-assay variability”. Preferably, the average coefficient of variation of intra-assay variability or inter-assay variability is less than 20%, more preferably less than 10%, more preferably less than 5%, more preferably less than 4%, more preferably less than 3%, more preferably less than 2%, and even more preferably less than 1%.

It has been determined that it is valuable to use the quadruplicate or triplicate test results to identify and eliminate data points that are statistical “outliers”; such data points are those that differ by a percentage greater, for example, than 3% of the average of all three or four values. Moreover, if more than one data point in a set of three or four is excluded by this procedure, then all data for the relevant constituent is discarded.

### Measurement of Gene Expression for a Constituent in the Panel

For measuring the amount of a particular RNA in a sample, methods known to one of ordinary skill in the art were used to extract and quantify transcribed RNA from a sample with respect to a constituent of a Gene Expression Panel (Precision Profile™). (See detailed protocols below. Also see PCT application publication number WO 98/24935 herein incorporated by reference for RNA analysis protocols). Briefly, RNA is extracted from a sample such as any tissue, body fluid, cell (e.g., circulating tumor cell) or culture medium in which a population of cells of a subject might be growing. For example, cells may be lysed and RNA eluted in a suitable solution in which to conduct a DNAse reaction. Subsequent to RNA extraction, first strand synthesis may be performed using a reverse transcriptase. Gene amplification, more specifically quantitative PCR assays, can then be conducted and the gene of interest calibrated against an internal marker such as 18S rRNA (Hirayama et al., Blood 92, 1998: 46-52). Any other endogenous marker can be used, such as 28S-25S rRNA and 5S rRNA. Samples are measured in multiple replicates, for example, 3 replicates. In an embodiment of the invention, quantitative PCR is performed using amplification, reporting agents and instruments such as those supplied commercially by Applied Biosystems (Foster City, Calif.). Given a defined efficiency of amplification of target transcripts, the point (e.g., cycle number) that signal from amplified target template is detectable may be directly related to the amount of specific message transcript in the measured sample. Similarly, other quantifiable signals such as fluorescence, enzyme activity, disintegrations per minute, absorbance, etc., when correlated to a known concentration of target templates (e.g., a reference standard curve) or normalized to a standard with limited variability can be used to quantify the number of target templates in an unknown sample.

Although not limited to amplification methods, quantitative gene expression techniques may utilize amplification of the target transcript. Alternatively or in combination with amplification of the target transcript, quantitation of the reporter signal for an internal marker generated by the exponential increase of amplified product may also be used. Amplification of the target template may be accomplished by isothermic gene amplification strategies or by gene amplification by thermal cycling such as PCR.

It is desirable to obtain a definable and reproducible correlation between the amplified target or reporter signal, i.e., internal marker, and the concentration of starting templates. It has been discovered that this objective can be achieved by careful attention to, for example, consistent primer-template ratios and a strict adherence to a narrow permissible level of experimental amplification efficiencies (for example 80.0 to 100% +/−5% relative efficiency, typically 90.0 to 100% +/−5% relative efficiency, more typically 95.0 to 100% +/−2%, and most typically 98 to 100% +/−1% relative efficiency). In determining gene expression levels with regard to a single Gene Expression Profile, it is necessary that all constituents of the panels, including endogenous controls, maintain similar amplification efficiencies, as defined herein, to permit accurate and precise relative measurements for each constituent. Amplification efficiencies are regarded as being “substantially similar”, for the purposes of this description and the following claims, if they differ by no more than approximately 10%, preferably by less than approximately 5%, more preferably by less than approximately 3%, and more preferably by less than approximately 1%. Measurement conditions are regarded as being “substantially repeatable, for the purposes of this description and the following claims, if they differ by no more than approximately +/−10% coefficient of variation (CV), preferably by less than approximately +/−5% CV, more preferably +/−2% CV. These constraints should be observed over the entire range of concentration levels to be measured associated with the relevant biological condition. While it is thus necessary for various embodiments herein to satisfy criteria that measurements are achieved under measurement conditions that are substantially repeatable and wherein specificity and efficiencies of amplification for all constituents are substantially similar, nevertheless, it is within the scope of the present invention as claimed herein to achieve such measurement conditions by adjusting assay results that do not satisfy these criteria,directly, in such a manner as to compensate for errors, so that the criteria are satisfied after suitable adjustment of assay results.

In practice, tests are run to assure that these conditions are satisfied. For example, the design of all primer-probe sets are done in house, experimentation is performed to determine which set gives the best performance. Even though primer-probe design can be enhanced using computer techniques known in the art, and notwithstanding common practice, it has been found that experimental validation is still useful. Moreover, in the course of experimental validation, the selected primer-probe combination is associated with a set of features:

The reverse primer should be complementary to the coding DNA strand. In one embodiment, the primer should be located across an intron-exon junction, with not more than four bases of the three-prime end of the reverse primer complementary to the proximal exon. (If more than four bases are complementary, then it would tend to competitively amplify genomic DNA.)

In an embodiment of the invention, the primer probe set should amplify cDNA of less than 110 bases in length and should not amplify, or generate fluorescent signal from, genomic DNA or transcripts or cDNA from related but biologically irrelevant loci.

A suitable target of the selected primer probe is first strand cDNA, which in one embodiment may be prepared from whole blood as follows:

(a) Use of Whole Blood for Assessment of Predicted Survivability and/or Survival Time.

Human blood is obtained by venipuncture and prepared for assay. Cells are lysed and nucleic acids, e.g., RNA, is stabilized and extracted by various standard means.

Nucleic acids, RNA and or DNA, are purified from cells, tissues or fluids of the test population of cells. RNA is preferentially obtained from the nucleic acid mix using a variety of standard procedures (or RNA Isolation Strategies, pp. 55-104, in RNA Methodologies, A laboratory guide for isolation and characterization, 2nd edition, 1998, Robert E. Farrell, Jr., Ed., Academic Press), e.g., using a filter-based RNA isolation system from Ambion (RNAqueous™, Phenol-free Total RNA Isolation Kit, Catalog #1912, version 9908; Austin, Tex.) or the PAXgene™ Blood RNA System (from Pre-Analytix).

(b) Amplification Strategies.

Specific RNAs are amplified using message specific primers or random primers. The specific primers are synthesized from data obtained from public databases (e.g., Unigene, National Center for Biotechnology Information, National Library of Medicine, Bethesda, Md.), including information from genomic and cDNA libraries obtained from humans and other animals. Primers are chosen to preferentially amplify from specific RNAs obtained from the test or indicator samples (see, for example, RT PCR, Chapter 15 in RNA Methodologies, A Laboratory Guide for Isolation and Characterization, 2nd edition, 1998, Robert E. Farrell, Jr., Ed., Academic Press; or Chapter 22 pp. 143-151, RNA Isolation and Characterization Protocols, Methods in Molecular Biology, Volume 86, 1998, R. Rapley and D. L. Manning Eds., Human Press, or Chapter 14 Statistical refinement of primer design parameters; or Chapter 5, pp. 55-72, PCR Applications: protocols for functional genomics, M. A. Innis, D. H. Gelfand and J. J. Sninsky, Eds., 1999, Academic Press). Amplifications are carried out in either isothermic conditions or using a thermal cycler (for example, a ABI 9600 or 9700 or 7900 obtained from Applied Biosystems, Foster City, Calif.; see Nucleic acid detection methods, pp. 1-24, in Molecular Methods for Virus Detection, D. L. Wiedbrauk and D. H., Farkas, Eds., 1995, Academic Press). Amplified nucleic acids are detected using fluorescent-tagged detection oligonucleotide probes (see, for example, Taqman™ PCR Reagent Kit, Protocol, part number 402823, Revision A, 1996, Applied Biosystems, Foster City Calif.) that are identified and synthesized from publicly known databases as described for the amplification primers.

For example, without limitation, amplified cDNA is detected and quantified using detection systems such as the ABI Prism® 7900 Sequence Detection System (Applied Biosystems (Foster City, Calif.)), the Cepheid SmartCycler® and Cepheid GeneXpert® Systems, the Fluidigm BioMark™ System, and the Roche LightCycler® 480 Real-Time PCR System. Amounts of specific RNAs contained in the test sample can be related to the relative quantity of fluorescence observed (see for example, Advances in Quantitative PCR Technology: 5′ Nuclease Assays, Y. S. Lie and C. J. Petropolus, Current Opinion in Biotechnology, 1998, 9:43-48, or Rapid Thermal Cycling and PCR Kinetics, pp. 211-229, chapter 14 in PCR applications: protocols for functional genomics, M. A. Innis, D. H. Gelfand and J. J. Sninsky, Eds., 1999, Academic Press). Examples of the procedure used with several of the above-mentioned detection systems are described below. In some embodiments, these procedures can be used for both whole blood RNA and RNA extracted from cultured cells (e.g., without limitation, CTCs, and CECs). In some embodiments, any tissue, body fluid, or cell(s) (e.g., circulating tumor cells (CTCs) or circulating endothelial cells (CECs)) may be used for ex vivo assessment of predicted survivability and/or survival time affected by an agent. Methods herein may also be applied using proteins where sensitive quantitative techniques, such as an Enzyme Linked ImmunoSorbent Assay (ELISA) or mass spectroscopy, are available and well-known in the art for measuring the amount of a protein constituent (see WO 98/24935 herein incorporated by reference).

An example of a procedure for the synthesis of first strand cDNA for use in PCR amplification is as follows:

Materials

1. Applied Biosystems TAQMAN Reverse Transcription Reagents Kit (P/N 808-0234). Kit Components: 10× TaqMan RT Buffer, 25 mM Magnesium chloride, deoxyNTPs mixture, Random Hexamers, RNase Inhibitor, MultiScribe Reverse Transcriptase (50 U/mL) (2) RNase/DNase free water (DEPC Treated Water from Ambion (P/N 9915G), or equivalent).

Methods

1. Place RNase Inhibitor and MultiScribe Reverse Transcriptase on ice immediately. All other reagents can be thawed at room temperature and then placed on ice.

2. Remove RNA samples from −80° C. freezer and thaw at room temperature and then place immediately on ice.

3. Prepare the following cocktail of Reverse Transcriptase Reagents for each 100 mL RT reaction (for multiple samples, prepare extra cocktail to allow for pipetting error):

| | 1 reaction (mL) | 11X, e.g. 10 samples (μL) |
|---|---|---|
| 10X RT Buffer | 10.0 | 110.0 |
| 25 mM MgCl₂ | 22.0 | 242.0 |
| dNTPs | 20.0 | 220.0 |
| Random Hexamers | 5.0 | 55.0 |
| RNAse Inhibitor | 2.0 | 22.0 |
| Reverse Transcriptase | 2.5 | 27.5 |
| Water | 18.5 | 203.5 |
| Total: | 80.0 | 880.0 (80 μL per sample) |

4. Bring each RNA sample to a total volume of 20 μL in a 1.5 mL microcentrifuge tube (for example, remove 10 μL RNA and dilute to 20 μL with RNase/DNase free water, for whole blood RNA use 20 μL total RNA) and add 80 μL RT reaction mix from step 5, 2, 3. Mix by pipetting up and down.

5. Incubate sample at room temperature for 10 minutes.

6. Incubate sample at 37° C. for 1 hour.

7. Incubate sample at 90° C. for 10 minutes.

8. Quick spin samples in microcentrifuge.

9. Place sample on ice if doing PCR immediately, otherwise store sample at −20° C. for future use.

10. PCR QC should be run on all RT samples using 18S and β-actin.

Following the synthesis of first strand cDNA, one particular embodiment of the approach for amplification of first strand cDNA by PCR, followed by detection and quantification of constituents of a Gene Expression Panel (Precision Profile™) is performed using the ABI Prism® 7900 Sequence Detection System as follows:

Materials

1. 20× Primer/Probe Mix for each gene of interest.

2. 20× Primer/Probe Mix for 18S endogenous control.

3. 2× Taqman Universal PCR Master Mix.

4. cDNA transcribed from RNA extracted from cells.

5. Applied Biosystems 96-Well Optical Reaction Plates.

6. Applied Biosystems Optical Caps, or optical-clear film.

7. Applied Biosystem Prism® 7700 or 7900 Sequence Detector.

Methods

1. Make stocks of each Primer/Probe mix containing the Primer/Probe for the gene of interest, Primer/Probe for 18S endogenous control, and 2× PCR Master Mix as follows. Make sufficient excess to allow for pipetting error e.g., approximately 10% excess. The following example illustrates a typical set up for one gene with quadruplicate samples testing two conditions (2 plates).

| | 1X (1 well) (μL) |
|---|---|
| 2X Master Mix | 7.5 |
| 20X 18S Primer/Probe Mix | 0.75 |
| 20X Gene of interest Primer/Probe Mix | 0.75 |
| Total | 9.0 |

2. Make stocks of cDNA targets by diluting 95 μL of cDNA into 2000 μL of water. The amount of cDNA is adjusted to give Ct values between 10 and 18, typically between 12 and 16.

3. Pipette 9 μL of Primer/Probe mix into the appropriate wells of an Applied Biosystems 384-Well Optical Reaction Plate.

4. Pipette 10 μL of cDNA stock solution into each well of the Applied Biosystems 384-Well Optical Reaction Plate.

5. Seal the plate with Applied Biosystems Optical Caps, or optical-clear film.

6. Analyze the plate on the ABI Prism® 7900 Sequence Detector.

In another embodiment of the invention, the use of the primer probe with the first strand cDNA as described above to permit measurement of constituents of a Gene Expression Panel (Precision Profile™) is performed using a QPCR assay on Cepheid SmartCycler® and GeneXpert® Instruments as follows:
I. To run a QPCR assay in duplicate on the Cepheid SmartCycler® instrument containing three target genes and one reference gene, the following procedure should be followed.

A. With 20× Primer/Probe Stocks.

Materials
1. SmartMix™-HM lyophilized Master Mix.
2. Molecular grade water.
3. 20× Primer/Probe Mix for the 18S endogenous control gene. The endogenous control gene will be dual labeled with VIC-MGB or equivalent.
4. 20× Primer/Probe Mix for each for target gene one, dual labeled with FAM-BHQ1 or equivalent.
5. 20× Primer/Probe Mix for each for target gene two, dual labeled with Texas Red-BHQ2 or equivalent.
6. 20× Primer/Probe Mix for each for target gene three, dual labeled with Alexa 647-BHQ3 or equivalent.
7. Tris buffer, pH 9.0
8. cDNA transcribed from RNA extracted from sample.
9. SmartCycler® 25 μL tube.
10. Cepheid SmartCycler® instrument.

Methods
1. For each cDNA sample to be investigated, add the following to a sterile 650 μL tube.

| | | |
|---|---|---|
| SmartMix ™-HM lyophilized Master Mix | 1 | bead |
| 20X 18S Primer/Probe Mix | 2.5 | μL |
| 20X Target Gene 1 Primer/Probe Mix | 2.5 | μL |
| 20X Target Gene 2 Primer/Probe Mix | 2.5 | μL |
| 20X Target Gene 3 Primer/Probe Mix | 2.5 | μL |
| Tris Buffer, pH 9.0 | 2.5 | μL |
| Sterile Water | 34.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18S reference gene CT value between 12 and 16.
3. Add 3 μL of the prepared cDNA sample to the reagent mixture bringing the total volume to 50 μL. Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler® tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.

B. With Lyophilized SmartBeads™.

Materials
1. SmartMix™-HM lyophilized Master Mix.
2. Molecular grade water.
3. SmartBeads™ containing the 18S endogenous control gene dual labeled with VIC-MGB or equivalent, and the three target genes, one dual labeled with FAM-BHQ1 or equivalent, one dual labeled with Texas Red-BHQ2 or equivalent and one dual labeled with Alexa 647-BHQ3 or equivalent.
4. Tris buffer, pH 9.0
5. cDNA transcribed from RNA extracted from sample.
6. SmartCycler® 25 μL tube.
7. Cepheid SmartCycler® instrument.

Methods
1. For each cDNA sample to be investigated, add the following to a sterile 650 μL tube.

| | | |
|---|---|---|
| SmartMix ™-HM lyophilized Master Mix | 1 | bead |
| SmartBear ™ containing four primer/probe sets | 1 | bead |
| Tris Buffer, pH 9.0 | 2.5 | μL |
| Sterile Water | 44.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18S reference gene CT value between 12 and 16.
3. Add 3 μL of the prepared cDNA sample to the reagent mixture bringing the total volume to 50 μL. Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler®tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.
II. To run a QPCR assay on the Cepheid GeneXpert® instrument containing three target genes and one reference gene, the following procedure should be followed. Note that to do duplicates, two self contained cartridges need to be loaded and run on the GeneXpert® instrument.

Materials
1. Cepheid GeneXpert® self contained cartridge preloaded with a lyophilized SmartMix™-HM master mix bead and a lyophilized SmartBead™ containing four primer/probe sets.
2. Molecular grade water, containing Tris buffer, pH 9.0.
3. Extraction and purification reagents.
4. Clinical sample (whole blood, RNA, etc.)
5. Cepheid GeneXpert® instrument.

Methods
1. Remove appropriate GeneXpert® self contained cartridge from packaging.
2. Fill appropriate chamber of self contained cartridge with molecular grade water with Tris buffer, pH 9.0.
3. Fill appropriate chambers of self contained cartridge with extraction and purification reagents.
4. Load aliquot of clinical sample into appropriate chamber of self contained cartridge.
5. Seal cartridge and load into GeneXpert® instrument.
6. Run the appropriate extraction and amplification protocol on the GeneXpert® and analyze the resultant data.

In yet another embodiment of the invention, the use of the primer probe with the first strand cDNA as described above to permit measurement of constituents of a Gene Expression Panel (Precision Profile™) is performed using a QPCR assay on the Roche LightCycler® 480 Real-Time PCR System as follows:

Materials
1. 20× Primer/Probe stock for the 18S endogenous control gene. The endogenous control gene may be dual labeled with either VIC-MGB or VIC-TAMRA.
2. 20× Primer/Probe stock for each target gene, dual labeled with either FAM-TAMRA or FAM-BHQ1.
3. 2× LightCycler® 490 Probes Master (master mix).
4. 1× cDNA sample stocks transcribed from RNA extracted from samples.
5. 1× TE buffer, pH 8.0.
6. LightCycler® 480 384-well plates.
7. Source MDx 24 gene Precision Profile™ 96-well intermediate plates.
8. RNase/DNase free 96-well plate.
9. 1.5 mL microcentrifuge tubes.
10. Beckman/Coulter Biomek® 3000 Laboratory Automation Workstation.
11. Velocity11 Bravo™ Liquid Handling Platform.
12. LightCycler® 480 Real-Time PCR System.

Methods
1. Remove a Source MDx 24 gene Precision Profile™ 96-well intermediate plate from the freezer, thaw and spin in a plate centrifuge.
2. Dilute four (4) 1× cDNA sample stocks in separate 1.5 mL microcentrifuge tubes with the total final volume for each of 540 μL.
3. Transfer the 4 diluted cDNA samples to an empty RNase/DNase free 96-well plate using the Biomek® 3000 Laboratory Automation Workstation.
4. Transfer the cDNA samples from the cDNA plate created in step 3 to the thawed and centrifuged Source MDx 24 gene Precision Profile™ 96-well intermediate plate using Biomek® 3000 Laboratory Automation Workstation. Seal the plate with a foil seal and spin in a plate centrifuge.
5. Transfer the contents of the cDNA-loaded Source MDx 24 gene Precision Profile™ 96-well intermediate plate to a new LightCycler® 480 384-well plate using the Bravo™ Liquid Handling Platform. Seal the 384-well plate with a LightCycler® 480 optical sealing foil and spin in a plate centrifuge for 1 minute at 2000 rpm.
6. Place the sealed in a dark 4° C. refrigerator for a minimum of 4 minutes.
7. Load the plate into the LightCycler® 480 Real-Time PCR System and start the LightCycler® 480 software. Chose the appropriate run parameters and start the run.
8. At the conclusion of the run, analyze the data and export the resulting CP values to the database.

In some instances, target gene FAM measurements may be beyond the detection limit of the particular platform instrument used to detect and quantify constituents of a Gene Expression Panel (Precision Profile™). To address the issue of “undetermined” gene expression measures as lack of expression for a particular gene, the detection limit may be reset and the “undetermined” constituents may be “flagged”. For example without limitation, the ABI Prism® 7900HT Sequence Detection System reports target gene FAM measurements that are beyond the detection limit of the instrument (>40 cycles) as “undetermined”. Detection Limit Reset is performed when at least 1 of 3 target gene FAM C_{T} replicates are not detected after 40 cycles and are designated as “undetermined”. “Undetermined” target gene FAM C_{T} replicates are re-set to 40 and flagged. C_{T} normalization (Δ C_{T}) and relative expression calculations that have used re-set FAM C_{T} values are also flagged.

### Baseline Profile Data Sets

The analyses of samples from single individuals and from large groups of individuals provide a library of profile data sets relating to a particular panel or series of panels. These profile data sets may be stored as records in a library for use as baseline profile data sets. As the term “baseline” suggests, the stored baseline profile data sets serve as comparators for providing a calibrated profile data set that is informative about the predicted response to immunotherapy, survivability and/or survival time, or the effect of a variable on (e.g., the effect of an therapeutic agent) on the predicted survivability and/or survival time of a subject. Baseline profile data sets may be stored in libraries and classified in a number of cross-referential ways. One form of classification may rely on the characteristics of the panels from which the data sets are derived. The libraries may also be accessed for records associated with a single subject or particular clinical trial. The classification of baseline profile data sets may further be annotated with medical information about a particular subject, a medical condition, and/or a particular agent.

The choice of a baseline profile data set for creating a calibrated profile data set is related to the response to immunotherapy, survivability and/or survival time to be evaluated, monitored, or predicted, as well as, the intended use of the calibrated panel (e.g., as to monitor the affect of a therapeutic agent on predicted survivability and/or survival time of a subject over time). It may be desirable to access baseline profile data sets from the same subject for whom a first profile data set is obtained or from different subject at varying times, exposures to stimuli, drugs or complex compounds; or may be derived from like or dissimilar populations or sets-of subjects.

The profile data set may arise from the same subject for which the first data set is obtained, where the sample is taken at a separate or similar time, a different or similar site or in a different or similar biological condition. For example, a sample may be taken before stimulation or after stimulation with an exogenous compound or substance, such as before or after therapeutic treatment. Alternatively the sample is taken before or include before or after a surgical procedure for melanoma. The profile data set obtained from the unstimulated sample may serve as a baseline profile data set for the sample taken after stimulation. The baseline data set may also be derived from a library containing profile data sets of a population or set of subjects having some defining characteristic or biological condition. The baseline profile data set may also correspond to some ex vivo or in vitro properties associated with an in vitro cell culture. The resultant calibrated profile data sets may then be stored as a record in a database or library along with or separate from the baseline profile data base and optionally the first profile data set al. though the first profile data set would normally become incorporated into a baseline profile data set under suitable classification criteria. The remarkable consistency of Gene Expression Profiles associated with predicted survivability and/or survival times makes it valuable to store profile data, which can be used, among other things for normative reference purposes. The normative reference can serve to indicate the degree to which a subject conforms to a given prediction (e.g., response to immunotherapy, survivability and/or survival time).

### Calibrated Data

Given the repeatability achieved in measurement of gene expression, described above in connection with “Gene Expression Panels” (Precision Profiles™) and “gene amplification”, it was concluded that where differences occur in measurement under such conditions, the differences are attributable to differences in biological condition. Thus, it has been found that calibrated profile data sets are highly reproducible in samples taken from the same individual under the same conditions. Similarly, it has been found that calibrated profile data sets are reproducible in samples that are repeatedly tested. Also, it has been found that in repeated instances wherein calibrated profile data sets are obtained when samples from a subject are exposed ex vivo to a compound, that they are comparable to calibrated profile data from a sample that has been exposed to a sample in vivo.

### Calculation of Calibrated Profile Data Sets and Computational Aids

The calibrated profile data set may be expressed in a spreadsheet or represented graphically for example, in a bar chart or tabular form but may also be expressed in a three dimensional representation. The function relating the baseline and profile data may be a ratio expressed as a logarithm. The constituent may be itemized on the x-axis and the logarithmic scale may be on the y-axis. Members of a calibrated data set may be expressed as a positive value representing a relative enhancement of gene expression or as a negative value representing a relative reduction in gene expression with respect to the baseline.

Each member of the calibrated profile data set should be reproducible within a range with respect to similar samples taken from the subject under similar conditions. For example, the calibrated profile data sets may be reproducible, within 20%, and typically within 10%. In accordance with embodiments of the invention, a pattern of increasing, decreasing and no change in relative gene expression from each of a plurality of gene loci examined in the Gene Expression Panel (Precision Profile™) may be used to prepare a calibrated profile set that is informative with regards to predicted response to immunotherapy, survivability and/or survival time of a subject or populations or sets of subjects or samples. Patterns of this nature may be used to identify likely candidates for a drug trial, used alone or in combination with other clinical indicators to be prognostic with respect to response to immunotherapy, predicted survivability and/or survival time or may be used to guide the development of a pharmaceutical or nutraceutical through manufacture, testing and marketing.

The numerical data obtained from quantitative gene expression and numerical data from calibrated gene expression relative to a baseline profile data set may be stored in databases or digital storage mediums and may be retrieved for purposes including managing patient health care or for conducting clinical trials or for characterizing a drug. The data may be transferred in physical or wireless networks via the World Wide Web, email, or internet access site for example or by hard copy so as to be collected and pooled from distant geographic sites.

The method also includes producing a calibrated profile data set for the panel, wherein each member of the calibrated profile data set is a function of a corresponding member of the first profile data set and a corresponding member of a baseline profile data set for the panel, and wherein the baseline profile data set is related to the response to immunotherapy, predicted survivability and/or survival time of a melanoma diagnosed subject, with the calibrated profile data set being a comparison between the first profile data set and the baseline profile data set, thereby providing evaluation of the predicted response to immunotherapy, survivability and/or survival time of a melanoma-diagnosed subject.

In yet other embodiments, the function is a mathematical function and is other than a simple difference, including a second function of the ratio of the corresponding member of first profile data set to the corresponding member of the baseline profile data set, or a logarithmic function. In such embodiments, the first sample is obtained and the first profile data set quantified at a first location, and the calibrated profile data set is produced using a network to access a database stored on a digital storage medium in a second location, wherein the database may be updated to reflect the first profile data set quantified from the sample. Additionally, using a network may include accessing a global computer network.

In an embodiment of the present invention, a descriptive record is stored in a single database or multiple databases where the stored data includes the raw gene expression data (first profile data set) prior to transformation by use of a baseline profile data set, as well as a record of the baseline profile data set used to generate the calibrated profile data set including for example, annotations regarding whether the baseline profile data set is derived from a particular Signature Panel and any other annotation that facilitates interpretation and use of the data.

Because the data is in a universal format, data handling may readily be done with a computer. The data is organized so as to provide an output optionally corresponding to a graphical representation of a calibrated data set.

The above described data storage on a computer may provide the information in a form that can be accessed by a user. Accordingly, the user may load the information onto a second access site including downloading the information. However, access may be restricted to users having a password or other security device so as to protect the medical records contained within. A feature of this embodiment of the invention is the ability of a user to add new or annotated records to the data set so the records become part of the biological information.

The various embodiments of the invention may be also implemented as a computer program product for use with a computer system. The product may include program code for deriving a first profile data set and for producing calibrated profiles. Such implementation may include a series of computer instructions fixed either on a tangible medium, such as a computer readable medium (for example, a diskette, CD-ROM, ROM, or fixed disk), or transmittable to a computer system via a modem or other interface device, such as a communications adapter coupled to a network. The network coupling may be for example, over optical or wired communications lines or via wireless techniques (for example, microwave, infrared or other transmission techniques) or some combination of these. The series of computer instructions preferably embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (for example, shrink wrapped software), preloaded with a computer system (for example, on system ROM or fixed disk), or distributed from a server or electronic bulletin board over a network (for example, the Internet or World Wide Web). In addition, a computer system is further provided including derivative modules for deriving a first data set and a calibration profile data set.

The calibration profile data sets in graphical or tabular form, the associated databases, and the calculated index or derived algorithm, together with information extracted from the panels, the databases, the data sets or the indices or algorithms are commodities that can be sold together or separately for a variety of purposes as described in WO 01/25473.

In other embodiments, a clinical indicator may be used to assess the survivability of a melanoma diagnosed subject by interpreting the calibrated profile data set in the context of at least one other clinical indicator, wherein the at least one other clinical indicator is selected from the group consisting of blood chemistry, (e.g., MART-1, Melan-A, tyrosinase, and microphthalmia transcription factor (Mitt) levels) X-ray or other radiological or metabolic imaging technique, molecular markers in the blood, other chemical assays, and physical findings.

### Index Construction

In combination, (i) the remarkable consistency of Gene Expression Profiles with respect to the predicted response to immunotherapy, survivability and/or survival time across a population or set of subjects or samples, or across a population of cells and (ii) the use of procedures that provide substantially reproducible measurement of constituents in a Gene Expression Panel (Precision Profile™) giving rise to a Gene Expression Profile, under measurement conditions wherein specificity and efficiencies of amplification for all constituents of the panel are substantially similar, make possible the use of an index that characterizes a Gene Expression Profile, and which therefore provides a predicted measurement of response to immunotherapy, survivability and/or survival time.

An index may be constructed using an index function that maps values in a Gene Expression Profile into a single value that is pertinent to the predicted response to immunotherapy, survivability and/or survival time of a subject. The values in a Gene Expression Profile are the amounts of each constituent of the Gene Expression Panel (Precision Profile™). These constituent amounts form a profile data set, and the index function generates a single value—the index—from the members of the profile data set.

The index function may conveniently be constructed as a linear sum of terms, each term being what is referred to herein as a “contribution function” of a member of the profile data set. For example, the contribution function may be a constant times a power of a member of the profile data set. So the index function would have the form

I=ΣCiMi^{P(i)},

where I is the index, Mi is the value of the member i of the profile data set, Ci is a constant, and P(i) is a power to which Mi is raised, the sum being formed for all integral values of i up to the number of members in the data set. We thus have a linear polynomial expression. The role of the coefficient Ci for a particular gene expression specifies whether a higher ΔCt value for this gene either increases (a positive Ci) or decreases (a lower value) the likelihood of melanoma, the ΔCt values of all other genes in the expression being held constant.

The values Ci and P(i) may be determined in a number of ways, so that the index I is informative of the predicted survivability and/or survival time of a subject. One way is to apply statistical techniques, such as latent class modeling, to the profile data sets to correlate clinical data or experimentally derived data, or other data pertinent to the predicted survivability and/or survival time. In this connection, for example, may be employed the software from Statistical Innovations, Belmont, Mass., called Latent Gold®. Alternatively, other simpler modeling techniques may be employed in a manner known in the art. The index function for predicting the survivability and/or survival time of a melanoma-diagnosed subject may be constructed, for example, in a manner that a greater degree of response to immunotherapy, survivability and/or survival time (as determined by the profile data set for the Precision Profile™ described herein (Table 1)) correlates with a large value of the index function.

Just as a baseline profile data set, discussed above, can be used to provide an appropriate normative reference, and can even be used to create a Calibrated profile data set, as discussed above, based on the normative reference, an index that characterizes a Gene Expression Profile can also be provided with a normative value of the index function used to create the index. This normative value can be determined with respect to a relevant population or set of subjects or samples or to a relevant population of cells, so that the index may be interpreted in relation to the normative value. The relevant population or set of subjects or samples, or relevant population of cells may have in common a property that is at least one of age range, gender, ethnicity, geographic location, nutritional history, medical condition (e.g., melanoma), clinical indicator, medication (e.g., chemotherapy or radiotherapy), physical activity, body mass, and environmental exposure.

As an example, for illustrative purposes only, the index can be constructed, in relation to a normative Gene Expression Profile for a population or set of melanoma subjects, in such a way that a reading of approximately 1 characterizes normative Gene Expression Profiles of healthy subjects. Let us further assume that the predicted survivability that is the subject of the index is “less than three years survival time”; a reading of 1 in this example thus corresponds to a Gene Expression Profile that matches the norm for melanoma subjects who will survive less than three years. A substantially higher reading then may identify a subject experiencing melanoma who is predicted to survive greater than three years. The use of 1 as identifying a normative value, however, is only one possible choice; another logical choice is to use 0 as identifying the normative value. With this choice, deviations in the index from zero can be indicated in standard deviation units (so that values lying between −1 and +1 encompass 90% of a normally distributed reference population or set of subjects. Since it was determined that Gene Expression Profile values (and accordingly constructed indices based on them) tend to be normally distributed, the 0-centered index constructed in this manner is highly informative. It therefore facilitates use of the index in diagnosis or prognosis of disease and setting objectives for treatment.

Still another embodiment is a method of providing an index pertinent to predicting the response to immunotherapy, survivability and/or survival time of melanoma-diagnosed subjects based on a first sample from the subject, the first sample providing a source of RNAs, the method comprising deriving from the first sample a profile data set, the profile data set including a plurality of members, each member being a quantitative measure of the amount of a distinct RNA constituent in a panel of constituents selected so that measurement of the constituents is indicative of the predicted response to immunotherapy, survivability and/or survival time of the subject, the panel including at least one constituent of any of the genes listed in the Precision Profile™ for Predicting Melanoma (Table 1). In deriving the profile data set, such measure for each constituent is achieved under measurement conditions that are substantially repeatable, at least one measure from the profile data set is applied to an index function that provides a mapping from at least one measure of the profile data set into one measure of the predicted survivability and/or survival time of a melanoma-diagnosed subject, so as to produce an index pertinent to the survivability and/or survival time of the subject.

### Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic or prognostic test, assay, or method concerns the ability of the test, assay, or method to distinguish between the survivability and/or survival times of subjects having melanoma is based on whether the subjects have an “effective amount” or a “significant alteration” in the levels of a cancer survivability associated gene. By “effective amount” or “significant alteration”, it is meant that the measurement of an appropriate number of cancer survivability associated gene (which may be one or more) is different than the predetermined cut-off point (or threshold value) for that cancer gene and therefore indicates that the subjects response to immunotherapy, survivability and/or survival time for which the cancer gene(s) is a determinant.

The difference in the level of cancer associated gene(s) between subjects that survive (i.e., alive) and subjects that do not survive (i.e., dead) is preferably statistically significant. As noted below, and without any limitation of the invention, achieving statistical significance, and thus the preferred analytical and clinical accuracy, generally, but not always, requires that combinations of several cancer survivability associated gene(s) be used together in panels and combined with mathematical algorithms in order to achieve a statistically significant predicted survivability and/or survival time associated gene index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

Using such statistics, an “acceptable degree of diagnostic or prognostic accuracy”, is herein defined as a test or assay (such as the test of the invention for determining an effective amount or a significant alteration of cancer survivability associated gene(s), which thereby indicates the predicted survivability and/or survival time of a melanoma-diagnosed subject) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic or prognostic accuracy”, it is meant a test or assay in which the AUC (area under the ROC curve for the test or assay) is at least 0.75, desirably at least 0.775, more desirably at least 0.800, preferably at least 0.825, more preferably at least 0.850, and most preferably at least 0.875.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for developing melanoma, and the bottom quartile comprising the group of subjects having the lowest relative risk for developing melanoma. Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for each quartile remain clinically useful are widely used as risk factors for a disease. Often such lower diagnostic accuracy tests must be combined with additional parameters in order to derive meaningful clinical thresholds for therapeutic intervention, as is done with the aforementioned global risk assessment indices.

A health economic utility function is yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic or prognostic accuracy are commonly used for continuous measures, when a disease category or risk category (such as those at risk for dying within a short period of time from advanced refractory and or relapsed melanoma, or those who may survive a long period of time with advanced refractory and/or relapsed melanoma) has not yet been clearly defined by the relevant medical societies and practice of medicine, where thresholds for therapeutic use are not yet established, or where there is no existing gold standard for diagnosis or prognosis of the condition For continuous measures of risk, measures of diagnostic or prognostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

In general, by defining the degree of diagnostic or prognostic accuracy, i.e., cut points on a ROC curve, defining an acceptable AUC value, and determining the acceptable ranges in relative concentration of what constitutes an effective amount of the cancer survivability associated gene(s) of the invention allows for one of skill in the art to use the cancer survivability associated gene(s) to identify, diagnose, or prognose subjects with a pre-determined level of predictability and performance.

Results from the cancer associated gene(s) indices thus derived can then be validated through their calibration with actual results, that is, by comparing the predicted versus observed rate of survivability and/or survival time in a given population, and the best predictive cancer associated gene(s) selected for and optimized through mathematical models of increased complexity. Many such formula may be used; beyond the simple non-linear transformations, such as logistic regression, of particular interest in this use of the present invention are structural and synactic classification algorithms, and methods of risk index construction, utilizing pattern recognition features, including established techniques such as the Kth-Nearest Neighbor, Boosting, Decision Trees, Neural Networks, Bayesian Networks, Support Vector Machines, and Hidden Markov Models, as well as other formula described herein.

Furthermore, the application of such techniques to panels of multiple cancer associated gene(s) is provided, as is the use of such combination to create single numerical “risk indices” or “risk scores” encompassing information from multiple cancer survivability associated gene(s) inputs. Individual cancer survivability associated gene(s) may also be included or excluded in the panel of cancer survivability associated gene(s) used in the calculation of the cancer survivability associated gene(s) indices so derived above, based on various measures of relative performance and calibration in validation, and employing through repetitive training methods such as forward, reverse, and stepwise selection, as well as with genetic algorithm approaches, with or without the use of constraints on the complexity of the resulting cancer survivability associated gene(s) indices.

The above measurements of diagnostic or prognostic accuracy for cancer associated gene(s) are only a few of the possible measurements of the clinical performance of the invention. It should be noted that the appropriateness of one measurement of clinical accuracy or another will vary based upon the clinical application, the population tested, and the clinical consequences of any potential misclassification of subjects. Other important aspects of the clinical and overall performance of the invention include the selection of cancer associated gene(s) so as to reduce overall cancer associated gene(s) variability (whether due to method (analytical) or biological (pre-analytical variability, for example, as in diurnal variation), or to the integration and analysis of results (post-analytical variability) into indices and cut-off ranges), to assess analyte stability or sample integrity, or to allow the use of differing sample matrices amongst blood, cells, serum, plasma, urine, etc.

### Kits

The invention also includes a melanoma survivability and/or response to treatment detection reagent. In some embodiments, the detection reagent is one or more nucleic acids that specifically identify one or more melanoma nucleic acids (e.g., any gene listed in Table 1, sometimes referred to herein as melanoma as associated genes or melanoma associated constituents) by having homologous nucleic acid sequences, such as oligonucleotide sequences, complementary to a portion of the melanoma genes nucleic acids or antibodies to proteins encoded by the melanoma gene nucleic acids packaged together in the form of a kit. The oligonucleotides can be fragments of the melanoma survivability genes. For example the oligonucleotides can be 200, 150, 100, 50, 25, 10 or less nucleotides in length.

The kit may contain in separate containers a nucleic acid or antibody (either already bound to a solid matrix or packaged separately with reagents for binding them to the matrix), control formulations (positive and/or negative), and/or a detectable label. The reagents may also include ancillary agents such as buffering agents and stabilizing agents, e.g., polysaccharides and the like. Instructions (i.e., written, tape, VCR, CD-ROM, etc.) for carrying out the assay may be included in the kit. The assay may for example be in the form of PCR, a Northern hybridization or a sandwich ELISA, as known in the art.

In another embodiment, melanoma survivability detection reagents can be immobilized on a solid matrix such as a porous strip to form at least one melanoma survivability gene detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of melanoma genes present in the sample. The detection sites may be configured in any suitably detectable shape and are typically in the shape of a bar or dot spanning the width of a test strip.

Alternatively, survivability detection reagents can be labeled (e.g., with one or more fluorescent dyes) and immobilized on lyophilized beads to form at least one melanoma gene detection site. The beads may also contain sites for negative and/or positive controls. Upon addition of the test sample, the number of sites displaying a detectable signal provides a quantitative indication of the amount of melanoma genes present in the sample.

Alternatively, the kit contains a nucleic acid substrate array comprising one or more nucleic acid sequences. The nucleic acids on the array specifically identify one or more nucleic acid sequences represented by melanoma genes (see Table 1). In various embodiments, the expression of 1, 2, 3, 4, 5, 6, 7, 8, 9, 10, 15, 20, 25, 40 or 50 or more of the sequences represented by melanoma genes (see Table 1) can be identified by virtue of binding to the array. The substrate array can be on, i.e., a solid substrate, i.e., a “chip” as described in U.S. Pat. No. 5,744,305. Alternatively, the substrate array can be a solution array, i.e., Luminex, Cyvera, Vitra and Quantum Dots' Mosaic.

The skilled artisan can routinely make antibodies, nucleic acid probes, i.e., oligonucleotides, aptamers, siRNAs, antisense oligonucleotides, against any of the melanoma genes listed in Table 1.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1

### Gene Expression Profiles for Predicting the Survivability of Advanced Refractory and/or Relapsed Melanoma Subjects-Training Dataset

The following study was conducted to investigate whether any of the genes (i.e., RNA-based transcripts) shown in the Precision Profile™ for Melanoma Survivability (Table 1), individually or when paired with one or more genes, are predictive of primary endpoints of melanoma progression (i.e., survival time).

Whole blood samples were obtained from a total of 218 patients (sometimes referred to herein as the “1008” patient population) who each met the following inclusion criteria: 1) histologically confirmed melanoma that was surgically incurable and either a) Stage III melanoma (AJCC 6^{th} Edition) including locally relapsed, in transit lesions or draining nodes, or b) Stage IV melanoma (M1a, M1b, M1c); 2) received prior treatment including at least one systemic therapy for treatment of metastatic disease (prior systemic regimen for the treatment of metastatic melonama must have contained IL-2, dacarbazine and/or temozolamide or interferon-α; patient must have received at least one cycle at full dose); 3) documented disease progression after the last dose of prior therapy (including patients whose disease progressed during previous treatment (refractory), recurred following previous treatment (relapsed) or patients who could not tolerate previous treatment due to unacceptable toxitiy and subsequently progressed); 4) at least one measurable lesion according to Response Evaluation Criteria in Solid Tumors (RECIST) (where measurable disease is defined as at least one lesion that can be accurately measured in at least one dimension with longest diameter=2.0 cm using conventional techniques or =1.0 cm with spiral CT scan; skin lesions documented by color photography must have a longest diameter of at least 1.0 cm; if the measurable disease is restricted to a solitary lesion, its neoplastic nature must be confirmed by cytology or histology; clinically detected lesions will only be considered measurable when they are superficial (eg, skin nodules) and the longest diameter is =2 cm; palpable lymph nodes >2.0 cm should be demonstrable by CT scan; tumor lesions that are situated in a previously irradiated area will be considered measurable if progression is documented following completion of radiation therapy); 5) ECOG performance status (PS) 0 or 1; 6) age 18 years or older; 7) adequate bone marrow, hepatic, and renal function determined within 14 days prior to enrollment, defined as: a) absolute neutrophil count=1.5×10⁹ cells/L; b) platelets=100×10⁹/L; c) hemoglobin=10 g/dL; d) aspartate and alanine aminotransferases (AST, ALT)=2.5×ULN (=5×ULN, if documented liver metastases are present); e) total bilirubin=2×ULN (except patients with documented Gilbert's syndrome); 0 serum creatinine=2.0 mg/dL or calculated creatinine clearance=60 mL/min; 8) serum lactic acid dehyrdrogenase (LDH)=2×ULN; 9) patients must have recovered from all prior treatment-related toxicities, to baseline status, or to NCI CTCAE (v 3.0) Grade of 0 or 1, except for toxicities not considered a safety risk such as alopecia or residual peripheral neuropathy resulting from prior systemic therapy. Post-surgical pain shall not be considered a basis for exclusion; and 10) must have been willing and able to provide written informed consent.

Any subjects that met the following criteria were excluded from the study: 1) diagnosed with melanoma of ocular origin (uveal melanoma); 2) received treatment for cancer, including immunotherapy, within one month prior to enrollment (dosing); 3) received any prior vaccine therapy for the treatment of melanoma within the last 6 months (if received last dose of vaccine prior to 6 months patient is eligible); 4) received any prior CTLA4-inhibiting agent; 5) history of, chronic autoimmune disease (eg, Addison's disease, multiple sclerosis, Graves disease, Hashimoto's thyroiditis, inflammatory bowel disease, psoriasis, rheumatoid arthritis, systemic lupus erythematosus, hypophysitis, etc.; active vitiligo or a history of vitiligo will not be a basis for exclusion); 6) known active or chronic viral hepatitis; 7) history of inflammatory bowel disease, celiac disease, or other chronic gastrointestinal conditions associated with diarrhea or current acute colitis of any origin; 8) history of uveitis or melanoma-associated retinopathy; 9) potential requirement for systemic corticosteroids or concurrent immunosuppressive drugs based on prior history or received systemic steroids within the last 4 weeks prior to enrollment (note: inhaled or topical steroids in standard doses were allowed); 10) dementia or significantly altered mental status that would prohibit the understanding or rendering of informed consent and compliance with the requirements of this protocol; 11) any serious, uncontrolled medical disorder or active infection, which would impair their ability to receive study treatment. (note that patients with evidence of Acquired Immunodeficiency Syndrome [AIDS] were excluded); 12) brain metastases (radiological documentation of absence of brain metastases at screening was required for all patients (note that a history of treated brain mets was acceptable); 13) history of other malignancies, except for adequately treated basal cell carcinoma or squamous cell skin cancer or carcinoma of cervix, unless the patient was disease-free for at least 5 years; 14) pregnancy or breast-feeding (female patients must be surgically sterile or be postmenopausal for two years, or must have agreed to use effective contraception during the period of treatment and 12 months after; all female patients with reproductive potential must have had a negative pregnancy test (serum/urine) within 72 hours prior to enrollment);

RNA was isolated from the whole blood samples obtained from the 218 patients using the PAXgene™ Blood RNA System (Pre-Analytix). Quantitative PCR assays were performed using custom primers and probes for the 169 targeted genes shown in Table 1 (i.e., the Precision Profile™ for Melanoma Survivability) to obtain gene expression measurements.

1, 2, 3 and 4-gene models yielding the best prediction of the survivability of advanced refractory and/or relapsed melanoma subjects were generated using a Cox-type survival analysis as described below.

### Cox-Type Survival Model:

When time from an initial (baseline) state to some event (e.g., death) is known, it is possible to examine the predictive relationship between the gene expressions and the time to the event (i.e., survival time). Survival analysis can be used to quantify and assess the effects of the genes in statistical models, typically which predict the hazard rate for each subject based on predictors such as the subjects' gene expressions and other risk factors. The hazard rate is the probability of the event occurring during the next time period t+1 given that it has not occurred as of time period t.

A Cox-type proportional hazards model was employed to examine the predictive relationship between gene expression (i.e., the genes shown in Table 1) and the time to the event (i.e., survival time). The genes enter directly as predictors in a log-linear model consisting of an intercept (the baseline hazard rate which may vary over time period t) plus other terms such as the gene expressions and other time constant or time varying predictors. For example, if multiple blood draws are available at different times leading to multiple expressions for a given gene, the gene can be included in the model as a time varying predictor. In such models, a significant gene effect means that subjects with a higher expression on that gene have a significantly higher (lower) probability of experiencing the event (e.g., death) in the next period t, than those with a lower expression but otherwise the same on the other risk factors in the model.

In these models, the parameter estimates can also be used to obtain predictions for the expected survival time. Survival models were developed based on gene expression data obtained from blood draws from 218 subjects diagnosed with advanced melanoma (stage 4), as previously described.

The genes were entered into the survival models in the following way:
1. Separate models were developed for each of the 169 genes shown in the Precision Profile™ for Melanoma Survivability (Table 1), with one of the genes included in each of these models (i.e., 1-gene models).
2. Separate models were developed for each gene pair(s) (i.e., 2-gene models).
3. Separate models were developed for each gene triple(s) (i.e., 3-gene models).
4. Separate models were developed for each gene quadruplet(s) (i.e., 4-gene models).

Final gene models summarized and interpreted were those for which all genes in the model were incrementally significant at the 0.05 level. Cox-type hazard ratio survival model analysis was performed based on overall survival time (i.e., from date of blood draw to death).

Post analysis (post survival model development), some time groupings were established to provide simple tables for examining the extent to which the models could distinguish between those who died <10 months, 10-12 months, and those still alive. Of the 218 subjects in the study, there were 103 patients that died within 10 months (47.2%), 25 patients that died between 10-12 months (11.5%), 88 patients that were alive after 12 months (40.4%), and 2 patients that were censored prior to 6 months (i.e., alive, but in the study less than 6 months (0.9%).

### Cox-Type Models for Overall Survival

Enumeration methods generated numerous multi-gene models for expected survival time. P-value and mean differences for all 169 genes shown in Table 1 were established. A listing of the p-values for all 169 1-gene models are shown in Table 2 (ranked by p-value). All possible 2-gene models were estimated on the N=218 melanoma subjects based on all 169 genes shown in Table 1 (14,196 combinations from 169 genes), yielding over 2,000 2-gene models for which both genes were incrementally statistically significant at the 0.05 level (as contributors to the 2-gene model). A listing of the statistically significant 2-gene models is shown in Table 3, sorted from high to low using the entropy R² value shown in the fourth column of the table. As shown in column 1 and column 2 of Table 3, the highest ranked, most statistically significant 2-gene model capable of predicting the survivability (i.e., alive or dead) of the “1008” melanoma subjects, in which both genes were incrementally statistically significant at the 0.05 level, includes CTSD and PLA2G7. Their respective p-values are shown in columns 5 and 6. The estimated co-efficients (“beta1” and “beta2”) for the 2-gene models shown in Table 3 are shown in columns 7 and 8. The estimated co-efficients can be used to construct a risk score “index” using the formula beta1*gene1+beta2*gene2, where “gene1” and “gene2” represent the delta C_{T} values for a given subject. The higher the risk score, the larger the hazard rate and the lower the expected survival time.

3-gene models were estimated using a select list of 64 of the targeted genes shown in Table 1. The 64 genes used to estimate all 3-gene models is shown in Table 4. Using these 64 select genes to estimate 3-gene models yielded 5,285 3-gene models for which all three genes were incrementally statistically significant at the 0.05 level (as contributors to the 3-gene model), 972 3-gene models for which all 3 genes were incrementally statistically significant at the 0.01 level, and 88 3-gene models for which all three genes were incrementally statistically significant at the 0.001 level. The 972 3-gene models for which all three genes were incrementally statistically significant at the 0.01 level are shown in Table 5. As shown in Table 5, the 3-gene model CTSD, PLA2G7 and TXNRD1, was the most statistically significant 3-gene model capable of predicting the survivability of melanoma subjects (i.e., alive or dead). This 3-gene model (CTSD, PLA2G7 and TXNRD1) was used to enumerate all possible 4-gene models. 166 models were enumerated. Limiting all p-values to <0.01 and entropy R²≧0.05 reduced the number of 4-gene models from 166 to 14. A listing of the 14 4-gene models that met the statistical criteria of p-value <0.01 and entropy R²≧0.05 is shown in Table 6. As shown in Table 6, the 4-gene model CTSD, PLA2G7, TXNRD1, and IRAK3 was the most statistically significant model capable of predicting the survivability of melanoma subjects (i.e., alive or dead). This 4-gene model correctly classifies 70% of those who died within the first 12 months and 69% of those who were alive after 12 months

The coefficients (rounded-off) of the 4-gene Cox model, CTSD, PLA2G7, TXNRD1 and IRAK3 were used to generate a risk score for each patient, which in turn was used to calculate expected survival time on an individual patient basis. The risk score calculation was defined as -2[(CTSD-TXNRD1)+(IRAK3-PLA2G7)]. Cut off points were used to establish low, medium and high risk groups.

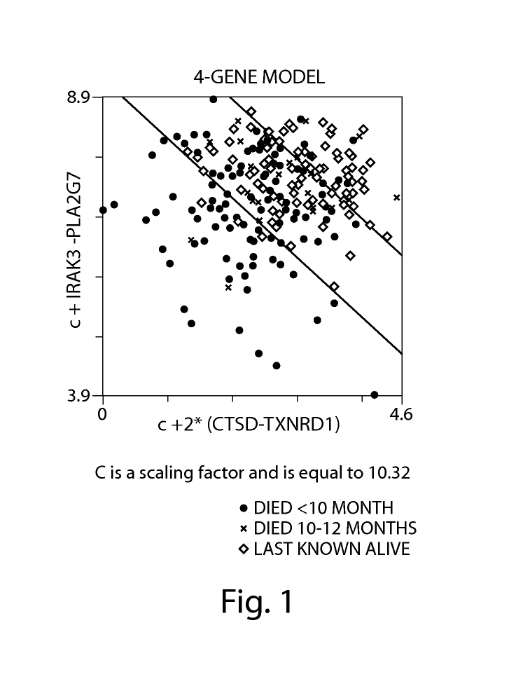

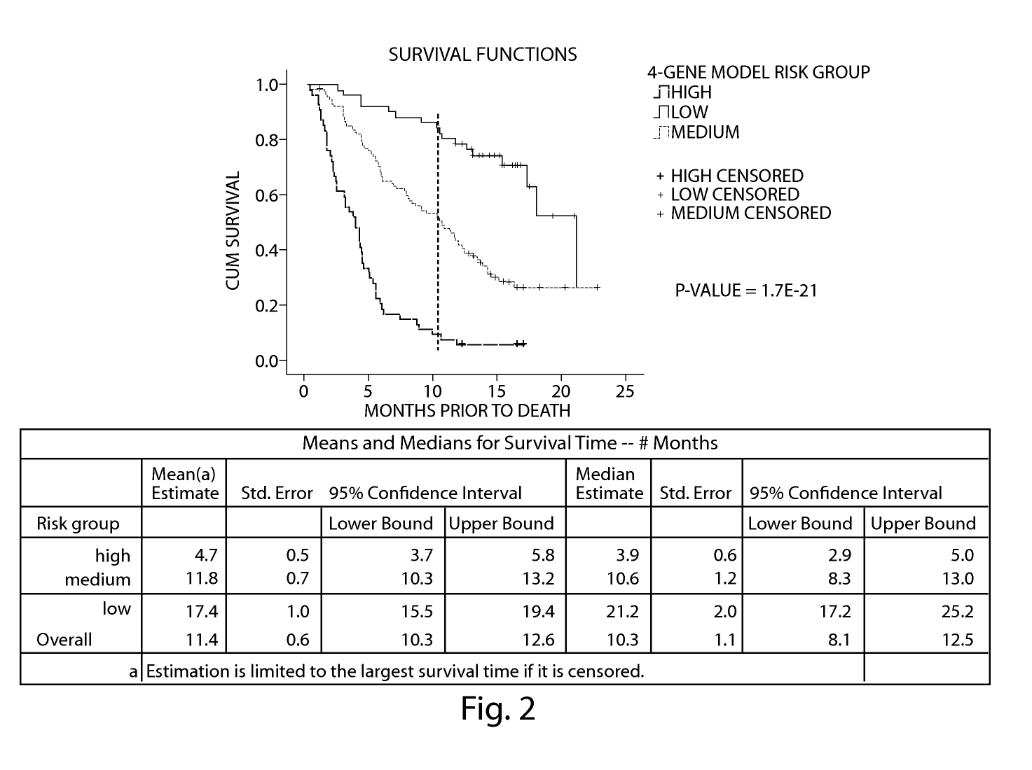

As shown in FIG. 1, the low risk (subjects above the upper line), medium risk (subjects in between the lines) and high risk groups (subjects below the lower line) as defined by the risk score -2[(CTSD-TXNRD1)+(IRAK3-PLA2G7)], provided a good prediction of survival time (i.e., there was a high correlation between risk group and survival time). As shown in Table 7, of the 52 subjects classified in the low risk group, 40 of the subjects (i.e., 76.9%) were last known as alive, and only 7 died <10 months (i.e., 13.5%). Of the 113 subjects classified in the medium risk group, 45 of the subjects were last known as alive (i.e., 39.8%) and 52 of the subjects died <10 months (i.e., 46%). Of the 54 subjects classified in the high risk group, only 3 were last known as alive (i.e., 5.6%), and 49 died <10 months (i.e., 90.7%). Kaplan-Meier assessment of the risk group results confirmed a strong prediction of survival time by the 4-gene risk model groups (FIG. 2).

### Example 2

### Latent Class Cox Models

For any kind of statistical model, including Cox models, one can estimate 1, 2, or 3 latent class models, for example, to see whether such models provide a better fit to the data as compared to e.g., a traditional Cox model. A latent class version of the 4-gene Cox model for overall survival (CTSD, PLA2G7, TXNRD1 and IRAK3) described in Example 1, revealed 2 latent classes: Class 1, with higher expected survival time (63% of subjects); and Class 2, with lower expected survival time (37% of subjects) (see Vermunt and Magidson, “LG-Syntax™ User's Guide: Manual for Latent GOLD® 4.5 Syntax Module”, Belmont MA: Statistical Innovations (2007).

The 4-gene risk score defined in Example 1 (-2[(CTSD-TXNRD1)+(IRAK3-PLA2G7)] was used to estimate the probability of an individual patient being in Class 1, and the distribution of expected survival time by month for each individual patient (see Vermunt and Magidson, “LG-Syntax™ User's Guide: Manual for Latent GOLD® 4.5 Syntax Module”, Belmont Mass.: Statistical Innovations (2007)). As shown in Table 8, 86% of the Class 1 patients survived at least 6 months compared to only 27% of Class 2 patients.

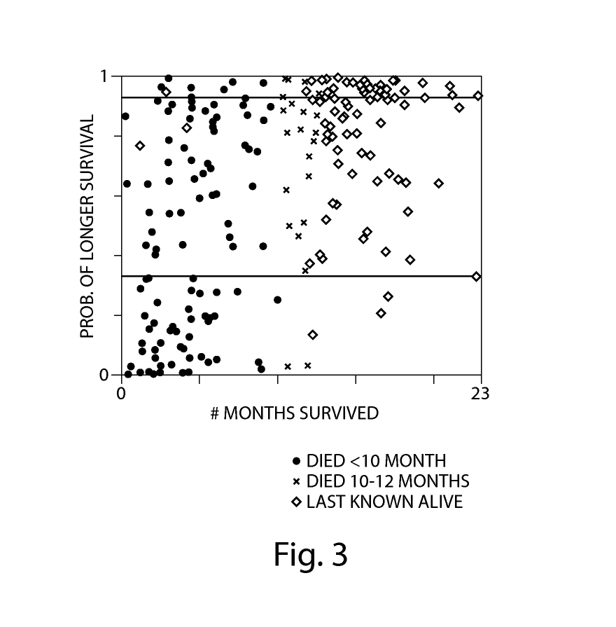

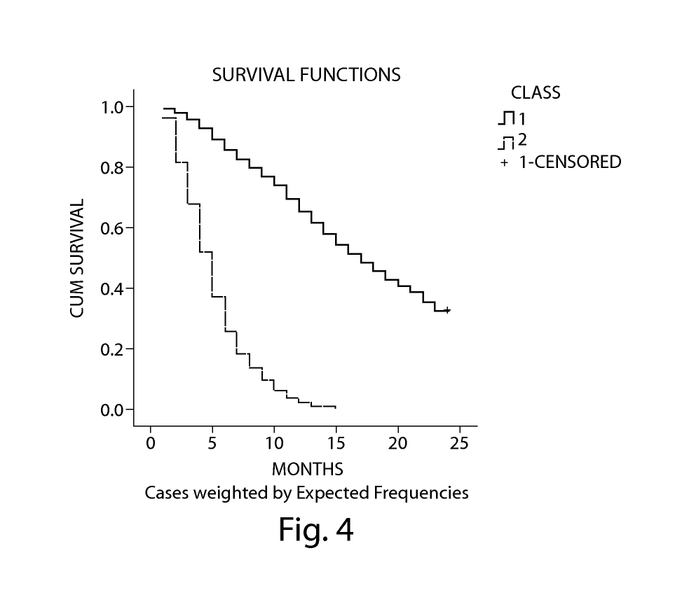

Cut off points were used to establish low, medium and high risk groups. As shown in FIG. 3, the low risk (subjects above the upper line), medium risk (subjects in between the lines) and high risk groups (subjects below the lower line) as defined by the risk score -2[(CTSD-TXNRD1)+(IRAK3-PLA2G7)], provided a good prediction of being in the longer surviving class. In other words, on an individual patient level, the predicted probability of being in the longer surviving latent class (i.e., Class 1), is predictive of survival. Subjects in the low risk group (i.e., above the upper line, had a 0.93 (or higher) probability of being in the longer surviving class; subjects in the medium risk group (between the lines) had between 0.33 an 0.93 probability of being in the longer surviving class; and subjects in the high risk group (below the lower line), had a 0.33 (or lower) probability of being in the longer surviving class. Kaplan-Meier assessment based on expected frequencies from the 2 latent classes confirmed a strong prediction of survival time by longer surviving latent class (i.e., Class 1) (FIG. 4).

These data support that Gene Expression Profiles generated with sufficient precision and calibration as described herein (1) can predict the survivability/and or survival time of melanoma-diagnosed subjects; (2) predict the probability of long term survivability and identify subsets of individuals among melanoma diagnosed subjects with a higher probability of long-term survivability based on their gene expression patterns; (3) may be used to monitor the affect of a therapeutic regimen on the survivability and/or survival time of melanoma diagnosed subjects; and (4) may be used to guide the medical management of a patient by adjusting therapy to bring one or more relevant Gene Expression Profiles closer to a target set of values, which may be normative values or other desired or achievable values.

Gene Expression Profiles are used for predicting the survivability and/or survival time of melanoma diagnosed subjects. Use of the algorithmic and statistical approaches discussed above to achieve such identification and to discriminate in such fashion is within the scope of various embodiments herein.

### Example 3

### Gene Expression Profiles for Predicting the Survivability of Advanced Refractory and/or Relapsed Melanoma Subjects-Test Dataset

The following study was conducted to determine whether the 2-gene models capable of predicting the survivability of melanoma subjects (i.e., alive or dead) could be validated using a separate population of melanoma patients.

Whole blood samples were obtained from a total of 264 patients (sometimes referred to herein as the “1009” patient population) who each met the following inclusion criteria: 1) Histologically confirmed melanoma that is not surgically curable and is either: a) Stage IV (AJCC 6th edition) or Stage IIIC (AJCC 6th edition) with N3 status for regional lymph nodes and in-transit or satellite lesions (note: patients with mucosal melanoma were not excluded; all HLA types were eligible); 2) Patients must have either had measurable disease or non-measurable disease which could be evaluated for objective response (measurable disease defined as: patient has at least one lesion that meets the following criteria: measurable lesions that can be accurately measured in at least one dimension; lesions on CT scan must have longest diameter ≧2.0 cm using conventional techniques or ≧1.0 cm with spiral CT scan. Skin lesions must have longest diameter at least 1.0 cm; clinically detected lesions must be superficial (eg, skin nodules), and the longest diameter must be ≧2.0 cm.; palpable lymph nodes ≧2.0 cm should be demonstrable by CT scan; if the measurable disease is restricted to a solitary lesion, its neoplastic nature must be confirmed by cytology or histology; tumor lesions that are situated in a previously irradiated area will be considered measurable only if progression is documented following completion of radiation therapy) (non-measurable disease defined as patients with non-measurable disease, i.e., without lesions that meet the above criteria for measurability; must have evidence of disease confirmed by pathology, i.e., needle aspirate/biopsy; patients with previously irradiated lesions must have documented progression or disease outside the radiation port); 3) ECOG performance status of 0 or 1; 4) age ≧18 years or older; 5) Adequate bone marrow, hepatic, and renal function determined within 14 days prior to randomization, defined as: a) Absolute neutrophil count ≧1.5×10⁹ cells/L; b) Platelets ≧100×10⁹/L; c) Hemoglobin ≧10 g/dL; d) Aspartate and alanine aminotransferases (AST, ALT) ≦2.5× Upper Limit of Normal (ULN), or ≦5×ULN, if documented liver metastases are present; e) Total serum bilirubin ≦1.5×ULN (except patients with documented Gilbert's syndrome); and f) Serum creatinine ≦2.0 mg/dL or calculated creatinine clearance ≧60 mL/min; 6) Serum lactic acid dehydrogenase (LDH) ≦2×ULN; 7) CT scan of the brain with contrast or MRI of the brain within 28 days of enrollment showing no evidence of brain metastases; 8) Patients must have recovered from all prior surgical or adjuvant (alpha-interferon) treatment-related toxicities, to baseline status, or a CTC Grade of 0 or 1, except for toxicities not considered a safety risk, such as alopecia; post-surgical pain was not considered a basis for exclusion; 9) Females of childbearing potential must have had a negative serum or urine pregnancy test within 14 days prior to randomization; females who underwent surgical sterilization or who were postmenopausal for at least 2 years were not considered to be of childbearing potential; 10) Females of childbearing potential and males who have not undergone surgical sterilization must have agreed to practice a form of effective contraception prior to entry into the study and for 6 months following the last dose of study drug; 11) Patient must have been willing and able to provide written informed consent.

Any subjects that met the following criteria were excluded from the study: 1) melanoma of ocular origin; 2) received any systemic therapy for metastatic melanoma except post-surgical adjuvant treatment with alpha-interferon for resected Stage II or Stage III disease; patients who received alpha-interferon must have been at least 30 days from the last dose, and must have documented tumor progression since the last dose (prior chemotherapy, biochemotherapy, cytokine therapy (other than alpha-interferon), or vaccine therapy was not allowed; prior intralesional injections and prior isolated limb perfusion therapy were not allowed; prior resection for Stage III or Stage IV disease was allowed as long as the patient had unresectable lesions at the time of randomization); 3) history of brain metastases; 4) received any prior CTLA4 inhibiting agent; 5) Patients previously randomized on this protocol; 6) history of chronic inflammatory or autoimmune disease (eg, Addison's disease, multiple sclerosis, Graves' disease, Hashimoto's thyroiditis, psoriasis, rheumatoid arthritis, systemic lupus erythematosus, hypophysitis, pituitary disorders, etc.; active vitiligo or a history of vitiligo was not a basis for exclusion); 7) history of uveitis or melanoma-associated retinopathy; 8) history of inflammatory bowel disease, celiac disease, or other chronic gastrointestinal conditions associated with diarrhea or bleeding, or current acute colitis of any origin; 9) history of hepatitis due to Hepatitis B virus or Hepatitis C virus; 10) any serious uncontrolled medical disorder or active infection that would impair the patient's ability to receive study treatment; 11) received an immunosuppressive dose of corticosteroids or other immunosuppressive medication (eg, methotrexate, rapamycin) within 30 days of randomization (patients with adrenal insufficiency could take up to 5 mg of prednisone or equivalent daily; topical and inhaled corticosteroids in standard doses were allowed); 12) history of other malignancy, except for adequately treated basal cell carcinoma or squamous cell skin cancer or carcinoma in situ of the cervix, unless the patient had been disease-free for at least 5 years; 13) breast-feeding; 14) dementia or significantly altered mental status that would prohibit the understanding or rendering of informed consent and compliance with the requirements of this protocol.

RNA was isolated from the whole blood samples obtained from the 264 patients using the PAXgene™ Blood RNA System. Quantitative PCR assays were performed using custom primers and probes for the 169 targeted genes shown in Table 1 (i.e., the Precision Profile™ for Melanoma Survivability) to obtain gene expression measurements.

All 2-gene models yielding the best prediction of the survivability of advanced refractory and/or relapsed melanoma subjects were estimated on the N=264 melanoma subjects using a Cox-type survival analysis as described in Example 1.

The results for all models where both genes are significant at the p<0.10 level, including the Wald p-values for each gene, are shown in Table 9 below, sorted from high to low using the entropy R² value shown in the fourth column of the table. As shown in column 1 and column 2 of Table 9, the highest ranked, most statistically significant 2-gene model capable of predicting the survivability of “1009” melanoma subjects (i.e., alive or dead) in which both genes were incrementally statistically significant at the 0.05 level includes CNKSR2 and IL1RN. Their respective p-values are shown in columns 5 and 6. The estimated co-efficients (“beta1” and “beta2”) for the 2-gene models shown in Table 9 are shown in columns 7 and 8. The estimated co-efficients can be used to construct a risk score “index” using the formula beta1*gene1+beta2*gene2, where “gene1” and “gene2” represent the delta C_{T} values for a given subject. The higher the risk score, the larger the hazard rate and the lower the expected survival time.

For most of the significant models shown in Table 9, it was observed that each gene became more incrementally significant in the 2-gene models. Without intending to be bound by any theory, in general modeling, a predictor often becomes less significant when another predictor is added to a model. Thus, the pattern seen in the significant models shown in Table 9, where each gene becomes more incrementally significant, is an unexpected and surprising result.

### Example 4

### Comparison of Training Dataset on the “1008” Melanoma Population and Test Dataset on the “1009” Melanoma Population

78% of the 2,200 2-gene models that were estimated on the “1008” melanoma population described in Example 1, where both genes were significant at the 0.05 level (as described in Example 1 and Table 3) also turned out to be significant at the 0.05 level on the “1009” melanoma population described in Example 3 and Table 9.

64.9% of 942 2-gene models that were estimated on the “1008” melanoma population, where both genes were significant at the 0.01 level but not at the 0.05 level, were significant at the 0.05 level in the “1009” melanoma population.

22.6% of 2-gene models that were estimated on the “1008 melanoma population, where at least 1 gene was non-significant at the 0.01 level, were still significant at the 0.05 level in the “1009” melanoma population.

This decreasing pattern (78%, 64.9%, 22.6%) is consistent with a successful validation. In other words, the 2 melanoma popultions are sufficiently similar so that the 2-gene models developed on the “1008” melanoma population are expected to also work (i.e., be cabaple of predicting the survivability of melanoma subjects) on the “1009” melanoma population.

Column 9 of Table 3 indicates which of the the 2-gene models that were estimated on the “1008” melanoma population described in Example 1 were validated on the “1009” melanoma population described in Example 2. Column 9 of Table 3 contains a ‘1’ if the 2-gene model validated on the “1009” data, and a ‘0’ if it did not validate. For the purpose of this analysis, validation means that the “1009” results also had significant p-values for each gene in the model, and the sign of the gene coefficients was the same as in the “1008” model.

4,073 of the 2-gene models that were estimated on the “1009” melanoma population had both genes significant at the 0.05 level. More models were significant among the “1009” data, compared to the “1008” data described in Example 1, which was expected, in part, due to a larger sample size and more deaths.

Additionally, many of the more significant models were observed to have an alternating +/− pattern for the gene coefficients (i.e., one beta value is positive, while the other beta value is negative). This was an unexpected and surprising observation. By chance, one might expect 50% of the significant models to show a +/− pattern (where the coefficient of 1 gene is ‘+’, while the coefficient of the other gene is ‘−’. However, 83% of the significant 2-gene models estimated on the “1008” melanoma population has a +/− pattern, and 81% of the significant 2-gene models estimated on the “1009” melanoma population has a +/− pattern. Thus, a prevalence of models with this special pattern was observed in both melanoma populations.

### Example 5

### Cox Models for Survival Also Predicts Response to Immunotherapy

A step wise inclusion Cox model was employed to examine the predictive relationship between gene expression (i.e., the genes shown in Table 1) and the time to the event (i.e., survival time or response to therapy).

Survival models as well as response to therapy were developed based on gene expression data obtained from pre and post treatment (immunotherapy) blood draws from 167 subjects diagnosed with advanced melanoma (stage 4), from the 1008 patient population. A listing of the statistically significant 1-gene, 2-gene, 3-gene, four-gene, and five-gene models is shown in Table 10. The two gene (CTLA4 and ST14) and the four gene model (CTLA4, ST14, IFI16 and ICAM) were selected for validation using the 1009 patient population. Both models validated with a p-val <0.05.

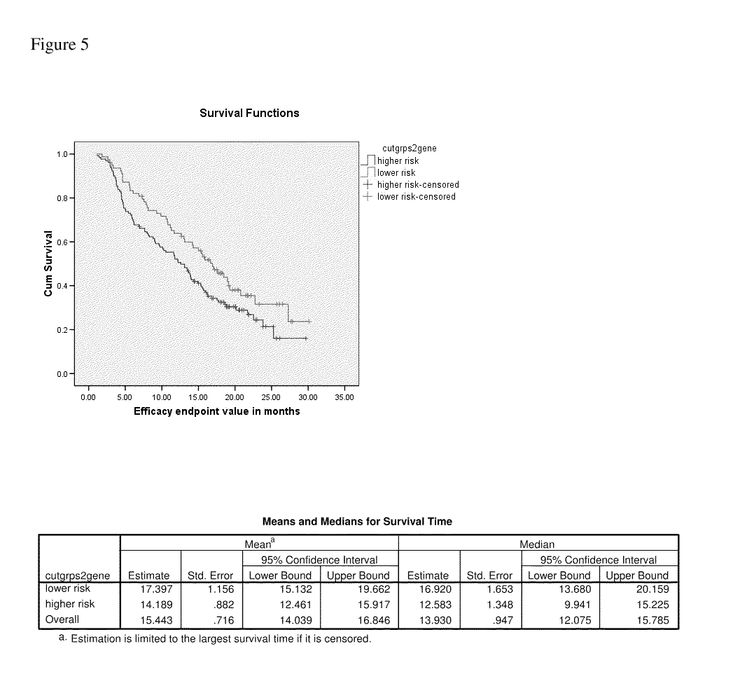

FIG. 5 shows a survival curve (Kaplan Meier) based on the 2-gene Cox-type model, CTLA4 and ST14) using the pre-specified risk score (0.46+042CTLA4-0.64ST14 and cut off points (0.03) which were established in the 1008 datasets yielded two risk groups (low and high)

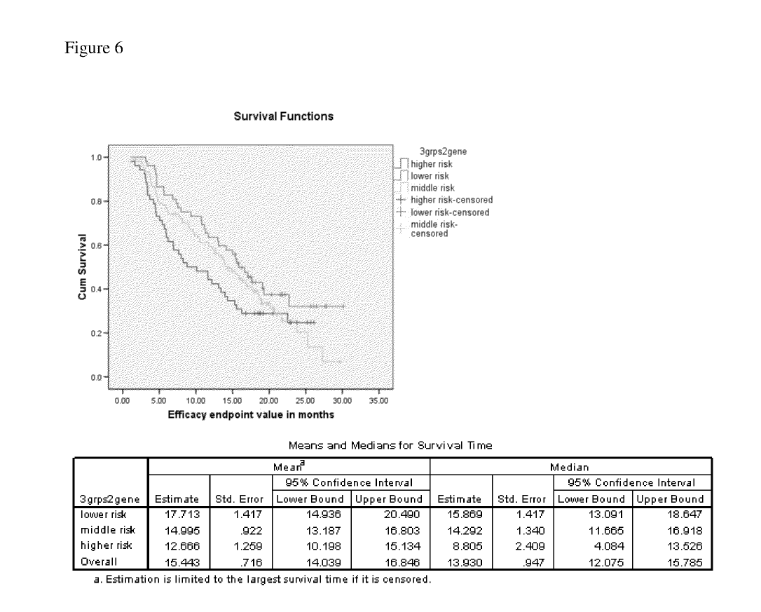

FIG. 6 shows a survival curve (Kaplan Meier) based on the 2-gene Cox-type model, CTLA4 and ST14 using prespecified percentile groups. The percentile groups were as follows Group 1, cases in the lowest score quartile (25%), Group 2, cases in the middle half (50%) and Group 3, cases in the highest score quartile (25%).

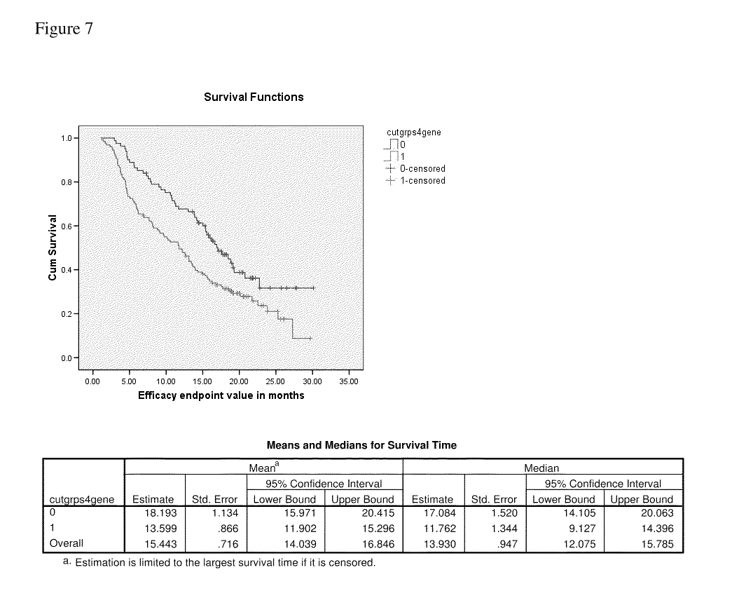

FIG. 7 shows a survival curve (Kaplan Meier) based on the 4-gene Cox-type model, CTLA4, ST14, IF116 and ICAM1 using the pre-specified risk score (0.63+045CTLA4-1.01ST14+0.75IFI16-014ICAM1 and cut off points (-0.31) which were established in the 1008 datasets yielded two risk groups (low and high)

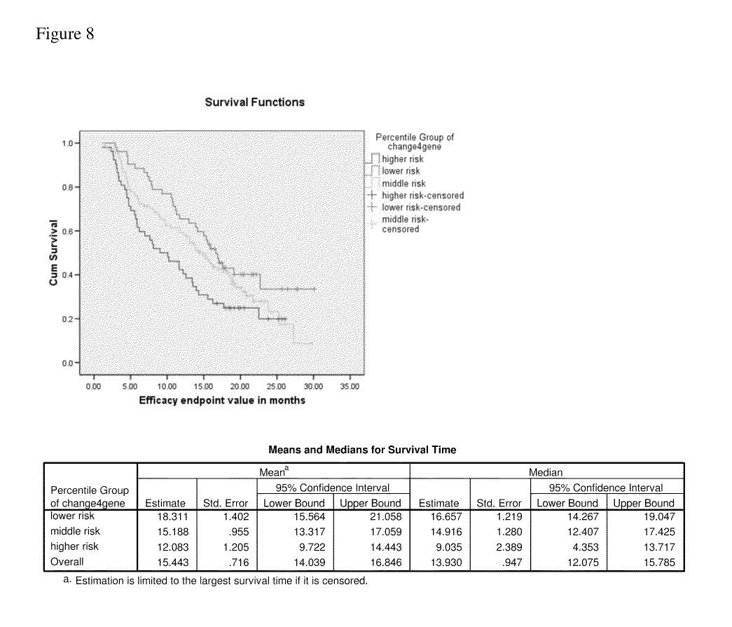

FIG. 8 shows a survival curve (Kaplan Meier) based on the n the 4-gene Cox-type model, CTLA4, ST14, IF116 and ICAM1 using prespecified percentile groups. The percentile groups were as follows Group 1, cases in the lowest score quartile (25%), Group 2, cases in the middle half (50%) and Group 3, cases in the highest score quartile (25%).

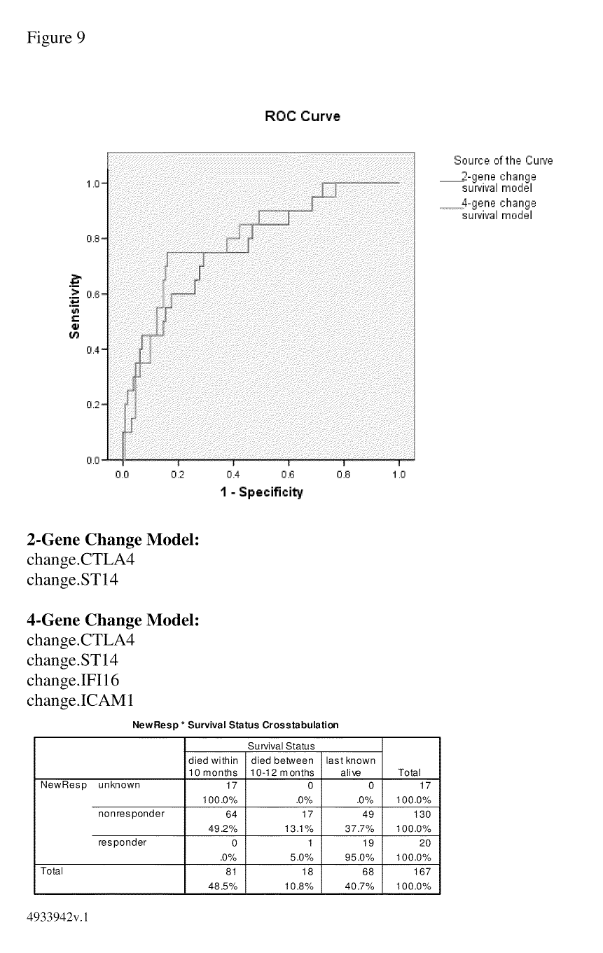

In addition to predicting survival, these models are also predictive of response to treatment. FIG. 9 shows a receiver operator curves (ROC) based on the 1008 population. As shown in the figure, the two gene and four gene Cox Survival Models based upon a change in the pre and post treatment gene expression is predictive of tumor response. Tables 11 and 12 shows the risk scores from the 2-gene and the 4-gene change model (post treatment—pre-treatment gene expression) measurements is predictive of tumor response. Importantly, the risk score from the 4 gene change model was also a predictor of tumor response in the 1009 population. (Table 13)

### Example 6

### Development of a Response to Immunotherapy Model that also Predicts Survival Using a New Step Down Algorithm

Using a new step down algorithm (K-Component) a seven gene response to immunotherapy treatment model was developed using pre treatment gene measurements from the 1009 patient population. (Described in U.S. Ser. No. 61/294,386, the contents of which is incorporated by reference its entirties). These seven genes in the model are LARGE, NFKB1, RBM5, HMGA1, BAX, TIMP, and HLADRA.

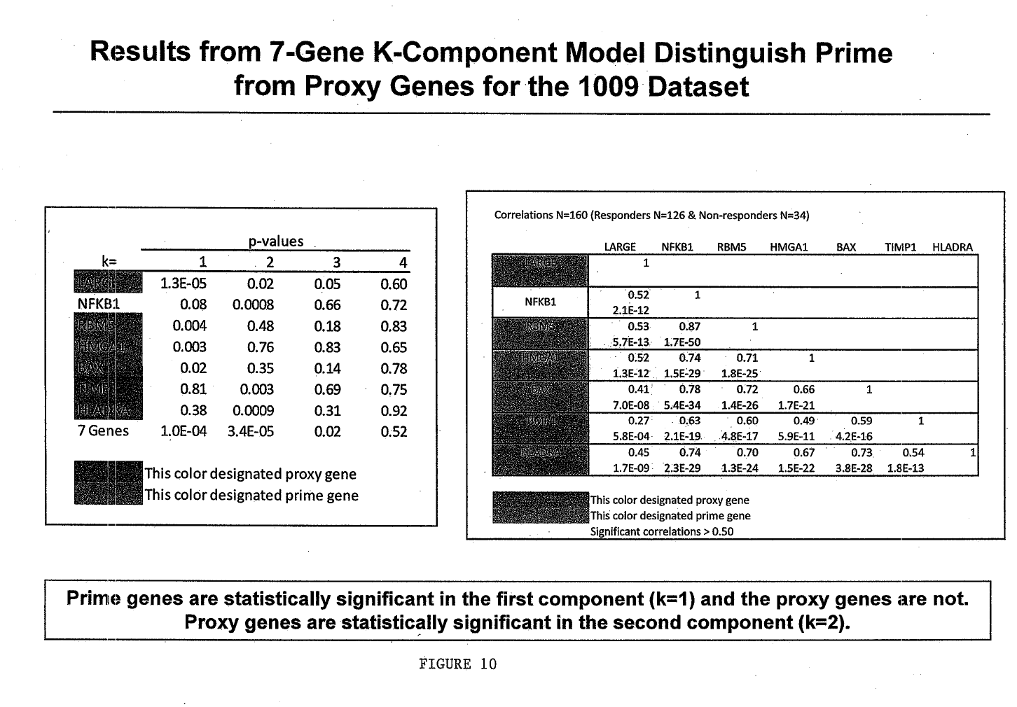

Briefly, this step down algorithm was based upon the observation that (i) one gene of the pair (referred to herein as a “Prime” gene) is significant when used separately in a 1-gene model; (ii) the other gene of the pair (referred to herein as a “Proxy” gene) is NOT significant when used separately in a 1-gene model; (iii) however, when the Proxy gene is included in a 2-gene model with the Prime gene, the Proxy gene significantly improves the predictive area under the ROC curve of the Prime gene alone; (iv) in the 2-gene model, one gene has a significant positive coefficient, while the other gene has a significant negative coefficient; and (v) the two genes have moderate to high positive correlation (>0.6). In the seven gee model, LARGE, RBM5, HMGA1 and BAX are prime genes and TIMP1 and HLADRA are proxy genes. See FIG. 10

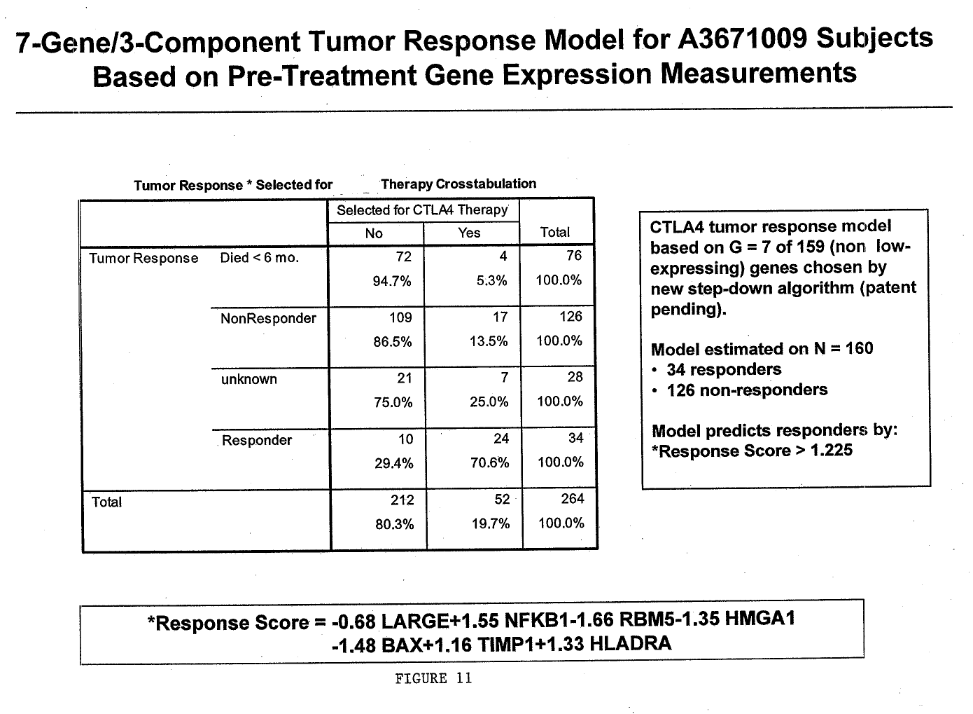

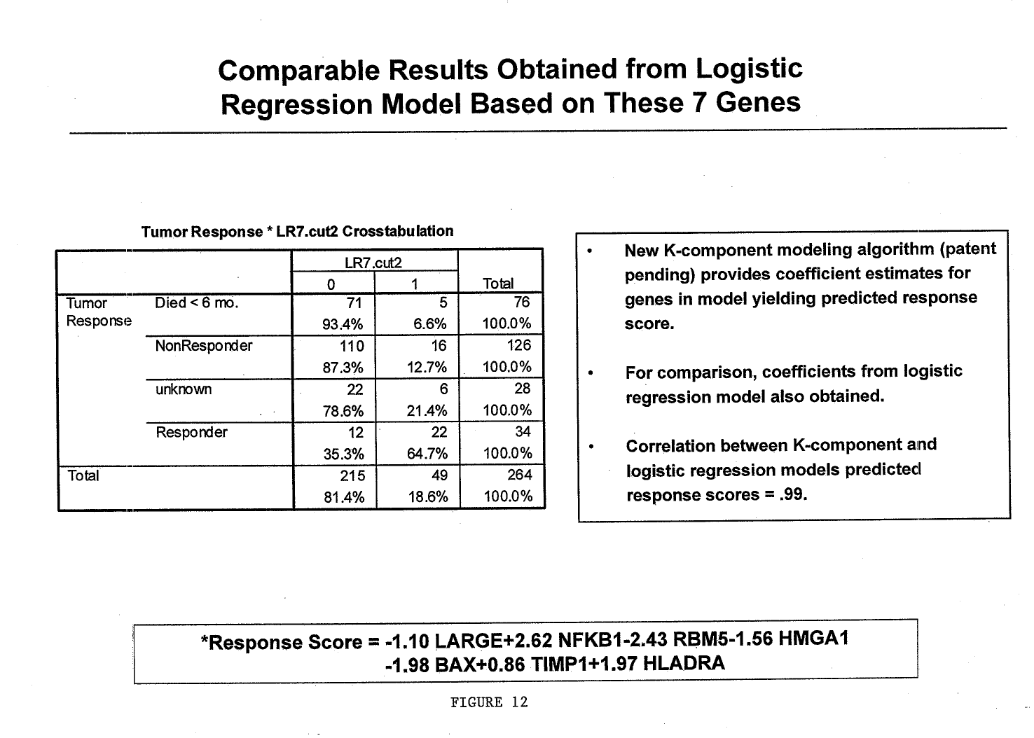

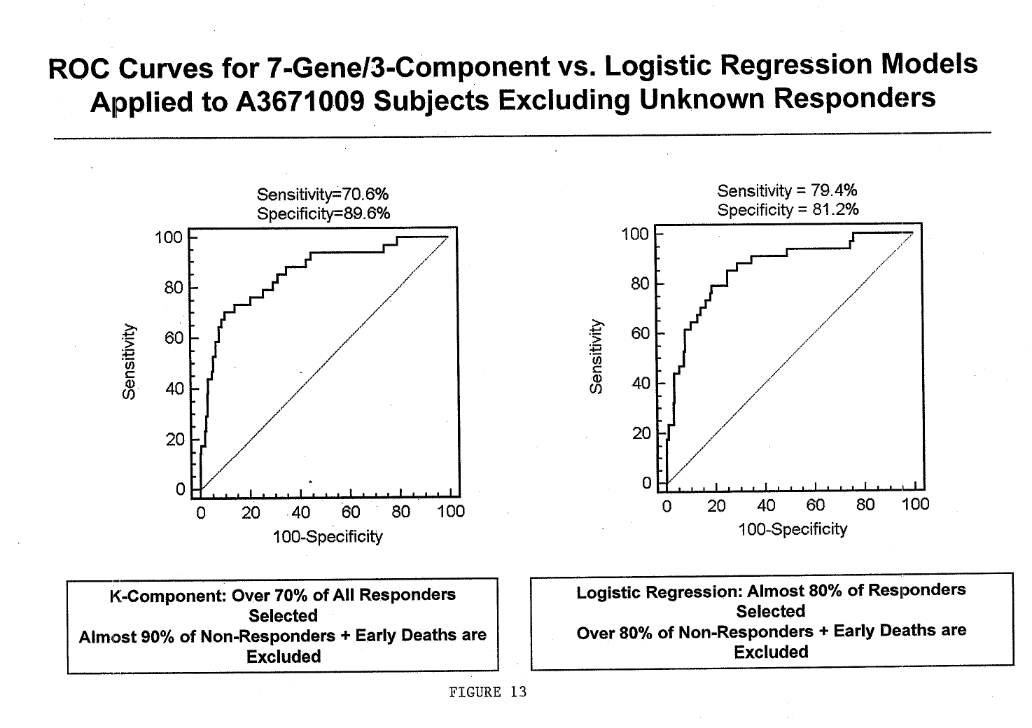

The model predicts responders by a response score of less than 1.225. (FIG. 11) In addition, comparable response prediction was obtained using a logistic regression model based upon these seven genes. (FIG. 12). As shown in FIG. 12, the correlation between the K-component model and the logistic regression models predicted a response score of 0.99. FIG. 13 shows ROC curves for the seven gene model versus logistic regression models for the 1009 subject population. As shown in FIG. 13, the 7 gene K-component models selected over 70% of all responders and almost 90% of all the non-responders. In contrast, the logistic regression model s selected almost 80% of all responders and over 80% of all the non-responders.

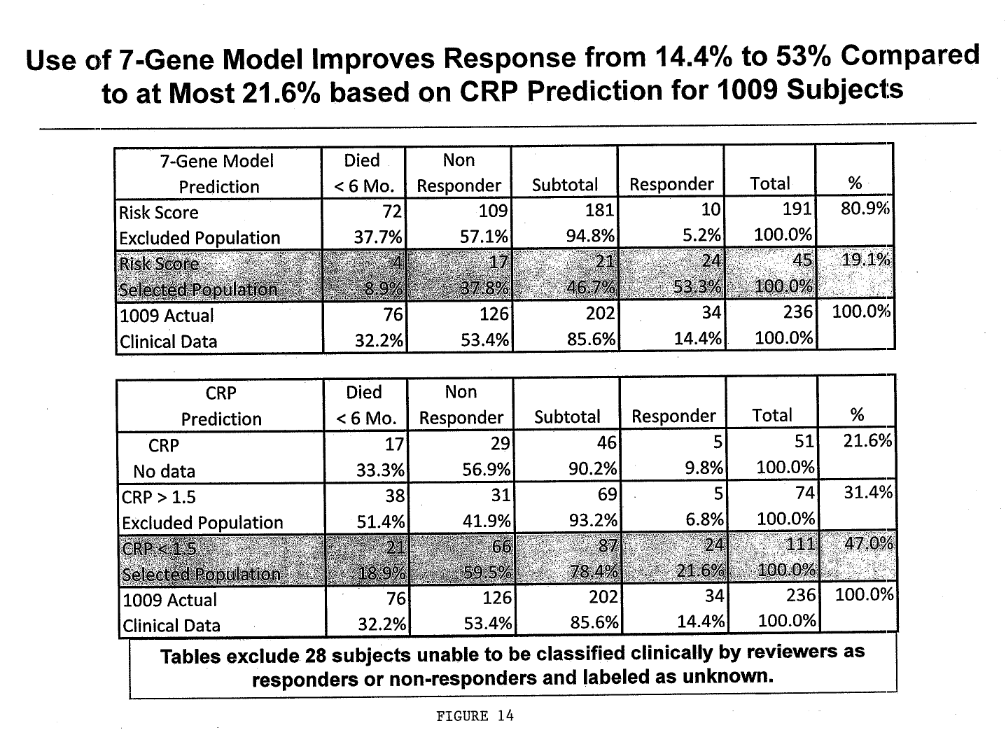

CRP levels are often used as a predictor of the progression of melanoma. As shown in FIG. 14, the seven gene model improves the prediction of response compared to CRP alone.

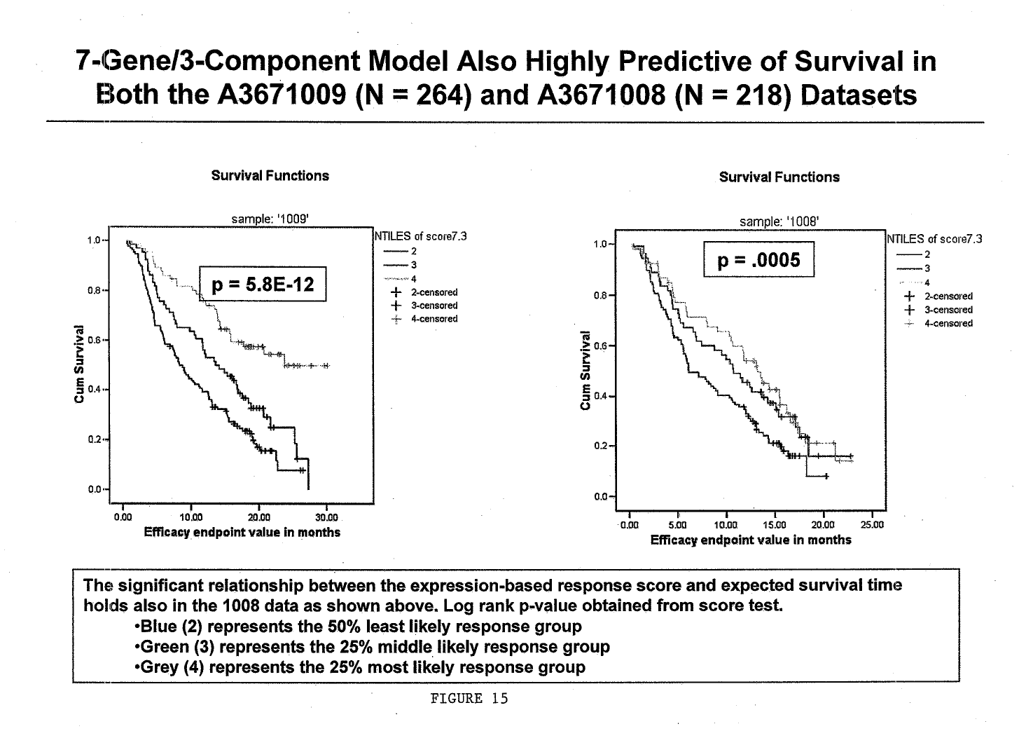

In addition, to predicting a subject's response to immunotherapy, the seven gene model is also highly predictive of survival. FIG. 15 shows a survival curve (Kaplan Meier) for both the 1008 and 1009 patient population.

To determine whether all seven genes in the response model were required to predict survival and or response, a backwards elimination Cox algorithm was applied the seven genes. The coefficients from each of these models are shown in Table 14. The performance of each of these models to predict 1) survival time of the 1009 population, 2) tumor response of the 1009 population and 3) survival time in the 1008 population is shown in Table 15. The results indicate that the four gene model (LARGE, NFKB1, BAX and TIMP1) is the strongest at predicting survival in the 1008 population and while this model significantly predicts tumor response to therapy the seven gene models is the strongest predictor.

The references listed below are hereby incorporated herein by reference.

### REFERENCES

Magidson, J. GOLDMineR User's Guide (1998). Belmont, Mass.: Statistical Innovations Inc.
Vermunt and Magidson (2005). Latent GOLD 4.0 Technical Guide, Belmont Mass.: Statistical Innovations.
Vermunt and Magidson (2007). LG-Syntax™ User's Guide: Manual for Latent GOLD® 4.5 Syntax Module, Belmont Mass.: Statistical Innovations.
Vermunt J. K. and J. Magidson. Latent Class Cluster Analysis in (2002) J. A. Hagenaars and A. L. McCutcheon (eds.), Applied Latent Class Analysis, 89-106. Cambridge: Cambridge University Press.
Magidson, J. “Maximum Likelihood Assessment of Clinical Trials Based on an Ordered Categorical Response.” (1996) Drug Information Journal, Maple Glen, Pa.: Drug Information Association, Vol. 30, No. 1, pp 143-170.

Lengthy table referenced here
US20110070582A1-20110324-T00001
Please refer to the end of the specification for access instructions.

Lengthy table referenced here
US20110070582A1-20110324-T00002
Please refer to the end of the specification for access instructions.

Lengthy table referenced here
US20110070582A1-20110324-T00003
Please refer to the end of the specification for access instructions.

Lengthy table referenced here
US20110070582A1-20110324-T00004
Please refer to the end of the specification for access instructions.

Lengthy table referenced here
US20110070582A1-20110324-T00005
Please refer to the end of the specification for access instructions.

Lengthy table referenced here
US20110070582A1-20110324-T00006
Please refer to the end of the specification for access instructions.

Lengthy table referenced here
US20110070582A1-20110324-T00007
Please refer to the end of the specification for access instructions.

Lengthy table referenced here
US20110070582A1-20110324-T00008
Please refer to the end of the specification for access instructions.

Lengthy table referenced here
US20110070582A1-20110324-T00009
Please refer to the end of the specification for access instructions.

Lengthy table referenced here
US20110070582A1-20110324-T00010
Please refer to the end of the specification for access instructions.

Lengthy table referenced here
US20110070582A1-20110324-T00011
Please refer to the end of the specification for access instructions.

Lengthy table referenced here
US20110070582A1-20110324-T00012
Please refer to the end of the specification for access instructions.

Lengthy table referenced here
US20110070582A1-20110324-T00013
Please refer to the end of the specification for access instructions.

Lengthy table referenced here
US20110070582A1-20110324-T00014
Please refer to the end of the specification for access instructions.

Lengthy table referenced here
US20110070582A1-20110324-T00015
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20110070582A1). An electronic copy of the table will also be available from the USPTO upon request and payment of the fee set forth in 37 CFR 1.19(b)(3).

What is claimed is:

1. A method for predicting the survivability of a melanoma-diagnosed subject based on a sample from the subject, the sample providing a source of RNAs, comprising:
a) determining a quantitative measure of the amount of:
i) CTSD, PLA2G7, TXNRD1 and IRAK3;
ii) at least two constituents according to any of the 2-gene models shown in Table 3 or 9;
iii) at least three constituents according to any of the 3-gene models shown in Table 5; or
iv) at least four constituents according to any of the 4-gene models shown in Table 6;
as distinct RNA constituents in the subject sample, wherein such measures are obtained under measurement conditions that are substantially repeatable and the constituents are selected so that measurement of the constituents enables prediction of the survivability or survival time of a melanoma-diagnosed subject; and
b) comparing the quantitative measure of the constituents in the subject sample to a reference value.

2. The method of claim 1, wherein at least 4 constituents are measured, and said constituents are CTSD, PLA2G7, TXNRD1 and IRAK3.

3. The method of claim 1, wherein said reference value is an index value.

4. The method of claim 1, wherein the sample is selected from the group consisting of blood, a blood fraction, a body fluid, cells and tissue.

5. The method of claim 1, wherein the measurement conditions that are substantially repeatable are within a degree of repeatability of better than ten percent.

6. The method of claim 1, wherein the measurement conditions that are substantially repeatable are within a degree of repeatability of better than five percent.

7. The method of claim 1, wherein the measurement conditions that are substantially repeatable are within a degree of repeatability of better than three percent.

8. The method of claim 1, wherein efficiencies of amplification for all constituents are substantially similar.

9. The method of claim 1, wherein the efficiency of amplification for all constituents is within ten percent.

10. The method of claim 1, wherein the efficiency of amplification for all constituents is within five percent.

11. The method of claim 1, wherein the efficiency of amplification for all constituents is within three percent.

12. A kit for predicting the survivability of a melanoma diagnosed subject, comprising at least one reagent for the detection or quantification of any constituent measured according to claim 1 and instructions for using the kit.

13. A method for predicting the response to immunotherapy and/or survivability of a melanoma-diagnosed subject based on a sample from the subject, the sample providing a source of RNAs, comprising:
a) determining a quantitative measure of the amount of:
i) CTLA4 and ST14 or
ii) LARGE, NFKB1, BAX and TIMP1
as distinct RNA constituents in the subject sample, wherein such measures are obtained under measurement conditions that are substantially repeatable and the constituents are selected so that measurement of the constituents enables prediction of response to therapy and/or the survivability or survival time of a melanoma-diagnosed subject; and
b) comparing the quantitative measure of the constituents in the subject sample to a reference value.

14. The method of claim 13, step a) i) further comprising determining a quantitative measure of the amount of IFI16 and ICAM.

15. The method of claim 13, step a) i) further comprising determining a quantitative measure of the amount of at least one additional gene selected from the group consisting RBM5, HMGA1 and HLADRA.

16. The method of claim 13, wherein said reference value is an index value.

17. The method of any of claims 13, wherein the sample is selected from the group consisting of blood, a blood fraction, a body fluid, cells and tissue.

18. The method of any of claims 13, wherein the measurement conditions that are substantially repeatable are within a degree of repeatability of better than ten percent.

19. The method of any of claims 13 wherein the measurement conditions that are substantially repeatable are within a degree of repeatability of better than five percent.

20. The method of any of claims 13, wherein the measurement conditions that are substantially repeatable are within a degree of repeatability of better than three percent.

21. The method of any of claims 13, wherein efficiencies of amplification for all constituents are substantially similar.

22. The method of any of claims 13, wherein the efficiency of amplification for all constituents is within ten percent.

23. The method of any of claims 13, wherein the efficiency of amplification for all constituents is within five percent.

24. The method of any of claims 13, wherein the efficiency of amplification for all constituents is within three percent.

25. A kit for predicting the response to immunotherapy and/or survivability of a melanoma diagnosed subject, comprising at least one reagent for the detection or quantification of any constituent measured according to a claim 13 and instructions for using the kit.
